(19) United States
(12) Patent Application Publication
(10) Pub. No.: US 2009/0233957 A1
(43) Pub. Date: Sep. 17, 2009

(54) (-)-BELOXEPIN AND METHODS FOR ITS SYNTHESIS AND USE

(75) Inventors: Bertrand Le Bourdonnec, East Fallowfield, PA (US); Roland E. Dolle, King of Prussia, PA (US)

Correspondence Address: DECHERT LLP, P.O. BOX 390460, MOUNTAIN VIEW, CA, 94039-0460, US

(73) Assignee: Adolor Corporation, Exton, PA (US)

(21) Appl. No.: 12/388,903

(22) Filed: Feb. 19, 2009

Related U.S. Application Data
(60) Provisional application No. 61/029,913, filed on Feb. 19, 2008.

(51) Int. Cl. A61K 31/4353; C07D 491/044; C07K 14/47; A61P 25/00

(52) U.S. Cl. 514/302; 546/61; 530/350

(57) ABSTRACT

This present disclosure provides compositions comprising (−)-beloxepin, methods for their synthesis and methods for their use.

### 1. CROSS REFERENCE TO RELATED APPLICATIONS

This application claims priority under 35 U.S.C. § 1.119(e) to provisional application No. 61/029,913 filed Feb. 19, 2008, the disclosure of which is incorporated herein by reference in its entirety.

### 2. STATEMENT REGARDING FEDERALLY SPONSORED RESEARCH

None.

### 3. PARTIES TO A JOINT RESEARCH AGREEMENT

None.

### 4. REFERENCE TO SEQUENCE LISTING, TABLE OR COMPUTER PROGRAM

None.

### 5. BACKGROUND

Acute and chronic pain of both nociceptive and non-nociceptive origin are disabling conditions that affect significant numbers of individuals. Pain is frequently characterized by increased sensitivity to normally non-noxious stimuli (allodynia) and/or painful stimuli (hyperalgesia). Although antidepressants such as norepinephrine and serotonin (5HT) reuptake inhibitors have been used as a first-line therapy for treating certain types of pain, for example, pain associated with diabetic neuropathy, postherpetic neuralgia, fibromyalgia, irritable bowel syndrome and interstitial cystitis, none of these therapies has proven to be universally effective. Despite the number of therapies available, significant numbers of individuals still suffer debilitating pain on a daily basis. Accordingly, there is a need in the art for additional compounds and regimens useful for treating pain, whether acute or chronic, or due to nociceptive or non-nociceptive origin.

### 6. SUMMARY

Racemic (±)-beloxepin, also known as “beloxepin,” “Org-4428” and “cis-1,2,3,4,4a,13b-hexahydro-2,10-dimethyldiben-[2,3:6,7]oxepino[4,5c]pyridine-4a-ol],” is a tetracyclic compound that underwent clinical evaluation as a potential antidepressant in the late 1990s. According to published reports, beloxepin is a highly specific inhibitor of noradrenaline reuptake in synaptosomes from rat and primate brain in in vitro assays, having greater than 100-fold less affinity for other monoamine carriers (i.e., serotonin and dopamine transporters), and no or very weak affinity for noradrenergic, histaminergic and cholinergic receptors (Sperling & Demling, 1997, Drugs of Today 33(2):95-102). It is also reported to have modest affinity for the 5HT_{2C} receptor (Claghorn & Lesem, 1996, Progress Drug Res 46:243-262).

In preclinical studies with animal models of depression, beloxepin was noted to exhibit antidepressant properties by offsetting acquired immobility behavior, reserpine-induced hypothermia, and conditioned avoidance behavior. In these tests, beloxepin did not cause sedation, motor impairment or other untoward side effects. Its profile on EEG-defined sleep/wake behavior is compatible with that of a nonsedative antidepressant with sleep-improving properties (Sperling & Demling, 1997, supra). Results of sleep studies in human volunteers have shown that beloxepin (25-400 mg) dose-dependently prolonged REM latency, both acutely and sub-chronically, and decreased total duration of nocturnal REM sleep as recorded by EEG (Van Bemmel et al., 1999, Neuropsychobiology 40(2):107-114). No sedation or other side effects were observed. Based on these studies, it was concluded that beloxepin may reduce sleep continuity in depressed patients and may improve the depth of sleep.

In a single-dose safety study, beloxepin displayed linear kinetics over a broad range, with a dose-independent tₘₐₓ of one to four hours and t_{1/2} of 11 to 15 hr following doses of 10 to 500 mg. Steady-state pharmacokinetic parameters obtained in healthy normal subjects, who participated in a multiple rising-dose safety and tolerance study, showed that at doses of 50 to 800 mg, tₘₐₓ was 1.17 hr and t_{1/2} varied from 12 to 14 hr. No important adverse effects were observed in healthy volunteers who received up to 800 mg/day of beloxepin. In a phase IIA study in patients hospitalized for depression, ⅔ of patients had a moderate to good response, based on HAMD score reduction (Claghorn & Lesem, 1996, supra).

In subsequent clinical trials, beloxepin exhibited insufficient efficacy for the treatment of major depression. Consequently further development of beloxepin was stopped (Paanakker et al., 1998, J. Pharm. Biomed. Anal. 16(6):981-989).

As will be discussed further herein, it has been surprisingly discovered by the present inventors that beloxepin is not a selective inhibitor of the norepinephrine transporter (“NET”), as reported in the literature. To the contrary, affinity testing with over 125 receptors, channels and transporters indicates that beloxepin binds with only modest affinity to the NET (Kᵢ=700 nM), and also binds with modest affinity to the 5HT_{2A}, 5HT_{2B} and 5HT_{2c} receptors (Kᵢ=440 nM, 1000 nM and 830 nM, respectively). In functional assays, beloxepin exhibited weak inhibition of norepinephrine reuptake (IC₅₀=130 nM) and antagonist activity at the 5HT_{2A}, 5HT_{2B} and 5HT_{2c} receptors (IC₅₀s of 5200 nM, >10,000 nM and >10,000 nM, respectively). Moreover, beloxepin exhibited only marginal affinity for the serotonin (27% inhibition at 10 μM) and dopamine (16% inhibition at 10 μM) transporters. Thus, it was surprisingly discovered that beloxepin, rather than being a selective NRI as reported in the literature, is a dual NET inhibitor/5HT_{2A,2B,2C} antagonist.

The chemical structure of beloxepin is illustrated below:

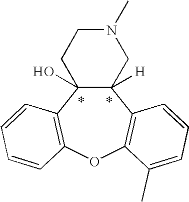

The OH and H substituents attached to the carbon atoms marked with asterisks are in the cis configuration with respect to one another. These carbon atoms are chiral. As a consequence, beloxepin is a racemic mixture of two cis enantiomers, a (+) enantiomer and a (−) enantiomer. The absolute configurations about the chiral carbons of the (+) and (−) enantiomers are unknown.

The biological activities of the (+) and (−) enantiomers of beloxepin have not been reported in the art. Studies carried out with these enantiomers by the present inventors reveals that they have distinct biological activities. Affinity and inhibitory data at the NET and 5HT_{2A}, 5HT_{2B} and 5HT_{2c} receptors for these enantiomers, as well as the data for racemic (±)-beloxepin are summarized in Table 1, below:

TABLE 1
Affinity and Activity of (+/−), (+), and (−)-beloxepin Data for Various
Transporters and Receptors

| | NET | | 5HT_{2A} | | 5HT_{2B} | | 5HT_{2C} | |
|---|---|---|---|---|---|---|---|---|
| | Kᵢ, nM | IC₅₀, nM | Kᵢ, nM | IC₅₀, nM | Kᵢ, nM | IC₅₀, nM | Kᵢ, nM | IC₅₀, nM |
| (±) | 700 | 130 | 440 | 5200 | 1000 | >10,000 | 830 | >10,000 |
| | | | | antagonist | | antagonist | | antagonist |
| (−) | 390 | 120 | >10,000 | nd | >10,000 | nd | >10,000 | nd |
| (+) | 2920 | 1200 | 97 | 1600 | 170 | 690 | 84 | 7200 |
| | | | | antagonist | | antagonist | | antagonist |

nd = not determined

The (−) enantiomer binds with approximately 8-fold higher affinity at the NET than the (+) enantiomer, while being devoid of any significant affinity at the 5HT_{2A}, 5HT_{2B} and 5HT_{2c} receptors. In stark contrast, the (+) enantiomer, which binds the NET with only weak affinity, displayed high affinity and antagonist activity at the 5HT_{2A}, 5HT_{2B} and 5HT_{2c} receptors. These data reveal that each of the dual biological activities discovered by the present inventors for beloxepin are contributed almost exclusively by a single enantiomer: the NRI activity by the (−) enantiomer, and the 5HT_{2A,2B,2C} antagonist activity by the (+) enantiomer. Thus, the present inventors have surprisingly discovered that beloxepin, rather than being a single compound with a single activity, is really three different compounds with three distinct biological activities: (i) racemic (±)-beloxepin, a dual NRI/5HT_{2A,2B,2C} antagonist; (ii) (+)-beloxepin, a 5HT_{2A,2B,2C} antagonist; and (iii) (−)-beloxepin, an NRI. All of these biological activities are known to correlate with therapeutic uses.

Accordingly, in one aspect, the present disclosure provides compositions comprising (−)-beloxepin and optionally one or more acceptable carriers, excipients or diluents. The (−)-beloxepin may be present in the composition as a non-racemic mixture enriched in the (−) enantiomer. In some embodiments, the (−)-beloxepin is substantially enantiomerically pure (−)-beloxepin. In some embodiments, the (−)-beloxepin is enantiomerically pure.

The (−)-beloxepin can be present in the composition in the form of the free base, or in the form of a salt. In some embodiments, the (−)-beloxepin is present in the form of a pharmaceutically acceptable acid addition salt.

The (−) beloxepin composition can be used in vitro or in vivo, as will be described in more detail below. When used in vivo, the composition can be formulated for administration to animals in veterinary contexts, or for administration to humans via virtually any route or mode of administration, including but not limited to, oral, topical, ocular, buccal, systemic, nasal, injection, transdermal, rectal, vaginal, inhalation or insufflation. In some embodiments, the composition is formulated for oral administration, for example, to humans.

Both selective and non-selective NRI compounds have proven effective in the treatment of a variety of diseases and disorders. It is expected that all of these diseases and disorders will likewise respond to treatment with (−)-beloxepin. Thus, in another aspect, the present disclosure provides methods of treating diseases and disorders responsive to treatment with NRI compounds. The methods generally comprise administering to a mammal, including a human, suffering from a disease or indication responsive to treatment with an NRI compound an amount of a (−)-beloxepin composition described herein effective to treat the disease or disorder. In some embodiments, the (−)-beloxepin composition comprises beloxepin that is enriched in the (−) enantiomer. In some embodiments, the beloxepin composition comprises substantially enantiomerically pure (−)-beloxepin. In some embodiments the beloxepin composition comprises enantiomerically pure (−)-beloxepin.

One important class of diseases or disorders known to be responsive to treatment with NRIs is mental disease. Specific examples of such mental diseases or disorders include, but are not limited to, the various mental diseases and indications classified in the Diagnostic and Statistic Manual of Mental Disorders IV (Text Revision 2000; referred to hereinafter as “DSM-IV”) as mood disorders (such as, for example, depression), anxiety disorders (such as, for example OCD), eating disorders, (such as, for example, anorexia nervosa and bulimia nervosa), impulse disorders (such as, for example, trichotillomania), sleep disorders (such as, for example, insomnia related to opioid withdrawal), personality disorders (such as, for example, ADHD), and somatoform disorders (such as certain types of pain). Another important class of diseases or indications known to be responsive to treatment with selective NRI compounds is pain, including both acute and chronic pain, whether nociceptive (for example somatic or visceral) or non-nociceptive (for example neuropathic or sympathetic) in origin (discussed further below). All of these diseases or disorders are expected to respond to treatment with various embodiments of the (−)-beloxepin compositions described herein.

The (−)-beloxepin composition can be administered alone, or it can be administered in combination with, or adjunctively to, one or more other drugs useful for treating indications responsive to NRI therapy and/or other indications. Specific non-limiting examples of drugs that can be used in combination with, or adjunctively to, the (−)-beloxepin compositions described herein in a regimen to treat diseases and/or disorders responsive to NRI therapy are provided in a later section.

In yet another aspect, the present disclosure provides methods of inhibiting the NE transporter. Inhibiting this transporter generally results in inhibition of reuptake of NE. The methods generally comprise contacting a NE transporter with an amount of (−)-beloxepin effective to inhibit the NET. In some embodiments, the method is carried out in the absence of (+)-beloxepin. In some embodiments, the NE transporter is contacted with a (−)-beloxepin composition as described herein. In some embodiments, the (−)-beloxepin composition comprises beloxepin that is enriched in the (−) enantiomer. In some embodiments, the (−)-beloxepin composition comprises substantially enantiomerically pure (−)-beloxepin. In some embodiments, the (−)-beloxepin composition comprises enantiomerically pure (−)-beloxepin.

The methods can be practiced in vitro with isolated transporters or cells that express the NE transporter, or in vivo as a therapeutic approach towards the treatment of diseases or disorders that are, at least in part, mediated by dysregulated reuptake of NE. Specific examples of diseases or disorders that are, at least in part, mediated by reuptake of NE include, but are not limited to, those listed above.

Historically, antidepressants including those that inhibit reuptake of NE (NRIs) and/or 5HT (SRIs) have been used as a first-line therapy for treating both acute and chronic pain that is either nociceptive or non-nociceptive in origin, for example, neuropathy, post-herpetic neuralgia (PHN), pain associated with fibromyalgia, pain associated with irritable bowel syndrome and interstitial cystitis (Sindrup and Jensen, 1999, Pain 83(3):389-400; Collins et al., 2000, J. Pain Symptom Management 20(6):449-458; Crowell et al., 2004, Current Opin. Investig. Drugs 5(7):736-742). A recent study systematically evaluated the relative activity at the NE and/or 5HT transporter required for maximal efficacy in rodent models of pain (Leventhal et al., 2007, J. Pharmacol. Exper. Ther. 320(3):1178-1185). The effects observed replicate those observed clinically for treating neuropathic pain conditions. Namely, compounds with greater affinity for the NE transporter are more effective at treating pain, and compounds with greater affinity for the 5HT transporter have limited efficacy (see, e.g., Max et al, 1992; N. Engl. J. Med. 326(19):1250-1256; Collins et al., 2000, supra). Indeed, in a double-blind, placebo-controlled head-to-head study comparing the tetracyclic NRI maprotiline and the SRI paroxetine, reduction in pain intensity was significantly greater for study completers randomized to maprotiline (45%) as compared to paroxetine (26%) or placebo (27%) (Atkinson et al., 1999, Pain 83(2):137-145).

In view of the moderate affinity of (−)-beloxepin for the NET (Kᵢ=390 nM), it would not have been predicted that this compound would be useful to treat pain. Notwithstanding that expectation, it has been surprisingly observed that, in experiments carried out by the applicants and reported herein, (−)-beloxepin exhibited robust therapeutic efficacy in a rodent model of pain. These data indicate that that (−)-beloxepin is ideally suited for the treatment of many different types of pain syndromes.

Accordingly, in yet another aspect, the present disclosure provides methods of treating pain in mammals, including humans. The methods generally comprise administering to a mammal suffering from pain, including a human, an amount of a (−)-beloxepin composition described herein effective to treat the pain. In some embodiments, the (−)-beloxepin composition comprises beloxepin that is enriched in (−) enantiomer. In some embodiments the (−)-beloxepin composition comprises substantially enantiomerically pure (−)-beloxepin. In some embodiments, the (−)-beloxepin composition comprises enantiomerically pure (−)-beloxepin.

The methods can be used to treat numerous different types of pain syndromes, including acute or chronic pain that is either nociceptive in origin (for example somatic or visceral) or non-nociceptive in origin (for example neuropathic or sympathetic). In some embodiments, the pain is nociceptive pain including, but not limited to, inflammatory pain such as that associated with inflammatory bowel syndrome (“IBS”) or rheumatoid arthritis, pain associated with cancer, and pain associated with osteoarthritis. In some embodiments the pain is non-nociceptive pain including, but not limited to, neuropathic pain such as post-herpetic neuralgia (PHN), trigeminal neuralgia, focal peripheral nerve injury, anesthesia clolorosa, central pain (for example, post-stroke pain, pain due to spinal cord injury or pain associated with multiple sclerosis), and peripheral neuropathy (for example, diabetic neuropathy, inherited neuropathy or other acquired neuropathies).

The (−)-beloxepin composition can be administered alone, or it can be administered in combination with, or adjunctively to, one or more other drugs useful for treating pain and/or other indications. Specific non-limiting examples of drugs that can be used in combination with, or adjunctively to, the (−)-beloxepin compositions described herein in a pain treatment or pain management regimen are provided in a later section.

Analogs of beloxepin have been reported in the art. For example, U.S. Pat. No. 4,977,158, the disclosure of which is incorporated herein by reference, discloses beloxepin analogs according to structural formula (I):

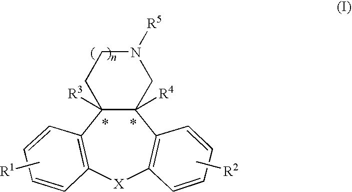

wherein:
n is 0 or 1;
X is O or S;
R¹ represents one or two identical or different substituents selected from H, OH, halogen, C₁-C₄ alkyl and C₁-C₄ alkoxy;
R² represents one or two identical or different substituents selected from H, OH, halogen, C₁-C₄ alkyl and C₁-C₄ alkoxy;
R³ and R⁴ are two substituents which are in the cis configuration and R³ is OH and R⁴ is H; and
R⁵ is H or C₁-C₄ alkyl.

It is expected that these beloxepin analogs comprise racemates and (+)-cis and (−)-cis enantiomers having distinct biological activities that correlate with the activities of the corresponding (±)-, (+)- and (−)-beloxepin isomers. Accordingly, the various enantiomers of the beloxepin analogs of structural formula (I) that correspond to the (−) enantiomer of beloxepin can be used in the compositions and methods described herein.

### 7. BRIEF DESCRIPTION OF THE FIGURES

FIG. 1 provides a graph demonstrating the antiallodynic effect of (+)- and (−)-beloxepin (30 mg/kg IP) in L5 SNL rats 8 days post surgery;

FIG. 2 provides a graph demonstrating the antiallodynic effect of (−)-beloxepin (30 mg/kg IP) in L5 SNL rats 14 days post surgery;

FIG. 3 provides a graph demonstrating the antiallodynic effect of orally administered (−)-beloxepin (60 mg/kg PO) in L5 SNL rats 7 days post surgery;

FIG. 4 provides a graph demonstrating the antiallodynic effect of orally administered (+)-beloxepin (60 mg/kg PO) in L5 SNL rats 14 days post surgery;

FIG. 5 provides a graph demonstrating the antiallodynic effect of (−)-beloxepin (30 mg/kg IP) in the rat hindpaw incisional model 24 hours post surgery;

FIG. 6 provides a graph demonstrating the antiallodynic effect of (+)-beloxepin (30 mg/kg IP) in the rat hindpaw incisional model 24 hours post surgery;

FIG. 7 provides a graph depicting the antinociceptive effects of (−)-beloxepin (30 mg/Kg) in the rat 50° C. hot plate model; and

FIG. 8 provides a graph depicting the antinociceptive effects of (+)-beloxepin (30 mg/Kg) in the rat 50° C. hot plate model.

### 8. DETAILED DESCRIPTION

### 8.1 (−)-Beloxepin Compounds and Compositions

The present disclosure concerns, among other things, compositions comprising the (−) enantiomer of racemic (±)-beloxepin. This racemate, which is also known as “beloxepin,” “Org-4428” and “cis-1,2,3,4,4a,13b-hexahydro-2,10-dimethyldiben-[2,3:6,7]oxepino[4,5c]pyridine-4a-ol],” is illustrated below:

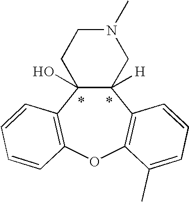

The OH and H substituents attached to the carbon atoms marked with asterisks are in the cis configuration with respect to one another. Since these carbons are chiral, this cis geometric isomer comprises two enantiomers, a (+) enantiomer and a (−) enantiomer. The absolute configurations about the chiral carbons of these (+) and (−) enantiomers are not presently known.

Analogs of beloxepin have been reported in the art. For example, U.S. Pat. No. 4,977,158, the disclosure of which is incorporated herein by reference, discloses beloxepin analogs according to structural formula (I):

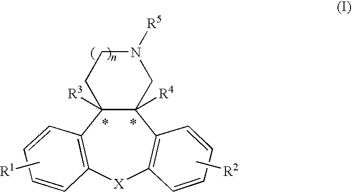

wherein:
n is 0 or 1;
X is O or S;
R¹ represents one or two identical or different substituents selected from H, OH, halogen, C₁-C₄ alkyl and C₁-C₄ alkoxy;
R² represents one or two identical or different substituents selected from H, OH, halogen, C₁-C₄ alkyl and C₁-C₄ alkoxy;
R³ and R⁴ are two substituents which are in the cis configuration and R³ is OH and R⁴ is H; and
R⁵ is H or C₁-C₄ alkyl.

Structural formula (I) is beloxepin when X is O, n is 1, R¹ and R⁴ are each H, R² is 2-methyl, R³ is OH and R⁵ is methyl. Although the various aspects of the instant disclosure are illustrated herein with (−)-beloxepin, it is expected that analogs of beloxepin according to structural formula (I), above, in which the configurations about the carbon atoms marked with asterisks relative to the oxepin ring are the same as those of (−)-beloxepin (referred to herein as “corresponding (−)-beloxepin analogs” or “corresponding enantiomers” or other grammatical equivalents) will have biological activities, and thus therapeutic uses, similar to those of (−)-beloxepin. Thus, the corresponding (−)-beloxepin analogs can also be used in the various compositions and methods described herein, and the various illustrative embodiments described for (−)-beloxepin apply also to the corresponding (−)-beloxepin analogs as if such embodiments were specifically described.

In the various (−)-beloxepin compositions described herein, the beloxepin can be present as a non-racemic mixture enriched in the (−) enantiomer, as the substantially enantiomerically pure (−) enantiomer or as the enantiomerically pure (−) enantiomer. In specific embodiments, the compositions comprise substantially enantiomerically pure (−)-beloxepin or enantiomerically pure (−)-beloxepin. Methods for synthesizing racemic beloxepin and isolating the (−) enantiomer via chiral separation are described in a later section.

As used herein, a racemic composition is “enriched” in a particular enantiomer when that enantiomer is present in excess over the other enantiomer, i.e., when that enantiomer comprises more than 50% of the total beloxepin in the composition. A composition that is enriched in a particular enantiomer will typically comprise at least about 60%, 70%, 80%, 90%, or even more, of the specified enantiomer. The amount of enrichment of a particular enantiomer can be confirmed using conventional analytical methods routinely used by those skilled in the art, including NMR spectroscopy in the presence of chiral shift reagents, gas chromatographic analysis using chiral columns, and high pressure liquid chromatographic analysis using chiral columns.

In some embodiments, a single enantiomer will be substantially free of the other enantiomer. By “substantially free of” is meant that the composition comprises less than about 10% of the specified undesired enantiomer, as established using conventional analytical methods routinely used by those of skill in the art, such as the methods mentioned above. In some embodiments, the amount of undesired enantiomer comprising the compound composition may be less than 10%, for example, 9%, 8%, 7%, 6%, 5%, 4%, 3%, 2%, 1% or even less. Enantiomerically enriched compound compositions that contain at least about 90% of a specified enantiomer are referred to herein as “substantially enantiomerically pure.” Thus, substantially enantiomerically pure compositions of chirally active compounds can contain in the range of at least about 90%, 91%, 92%, 93%, 94%, 95%, 96% or 97%, or even more (including any amount falling with the range of about 90-100%) of a specified enantiomer. Compositions of chirally active compounds that contain at least about 98% of a specified enantiomer are referred to herein as “enantiomerically pure.” Thus, enantiomerically pure compositions of chirally active compounds can contain in the range of at least about 98%, 99%, or even more (including any amount falling with the range of about 98-100%) of a specified enantiomer.

Depending upon the intended use, the (−)-beloxepin can be present in the composition as the free base, or in the form of a salt, for example, an acid additional salt. In some embodiments, the (−)-beloxepin is present in the composition in the form of a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (e.g., hydrochloric acid, hydrobromic acid, hydriodic, etc.), sulfuric acid, nitric acid, phosphoric acid and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzic acid, cinnamic acid, mandelic acid, alkylsulfonic acids (e.g., methanesulfonic acid, ethanesulfonic acid, 1,2-ethane-disulfonic acid, 2-hydroxyethanesulfonic acid, etc.), arylsulfonic acids (e.g., benzenesulfonic acid, 4-chlorobenzenesulfonic acid, 2-naphthalenesulfonic acid, 4-tuluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

In some embodiments, the (−)-beloxepin is present in the composition as the free base.

### 8.2 Methods of Synthesis

Beloxepin can be synthesized or prepared using methods described in the literature, for example, beloxepin can be synthesized as described in U.S. Pat. No. 4,977,158, the disclosure of which is incorporated herein by reference, and the (+) and (−) enantiomers isolated by conventional chiral chromatography (see, e.g., Chiral Separation Techniques: A Practical Approach, 2nd ed., Wiley-VCH, Weinheim, 2001). Beloxepin analogs can also be synthesized using the methods described in U.S. Pat. No. 4,977,158 and the corresponding (+) and (−) enantiomers isolated by conventional chiral chromatography.

A specific method for synthesizing racemic beloxepin which can be routinely adapted to synthesize racemic beloxepin analogs, and from which the corresponding (+) and (−) enantiomers can be isolated is illustrated in Scheme 1, below:

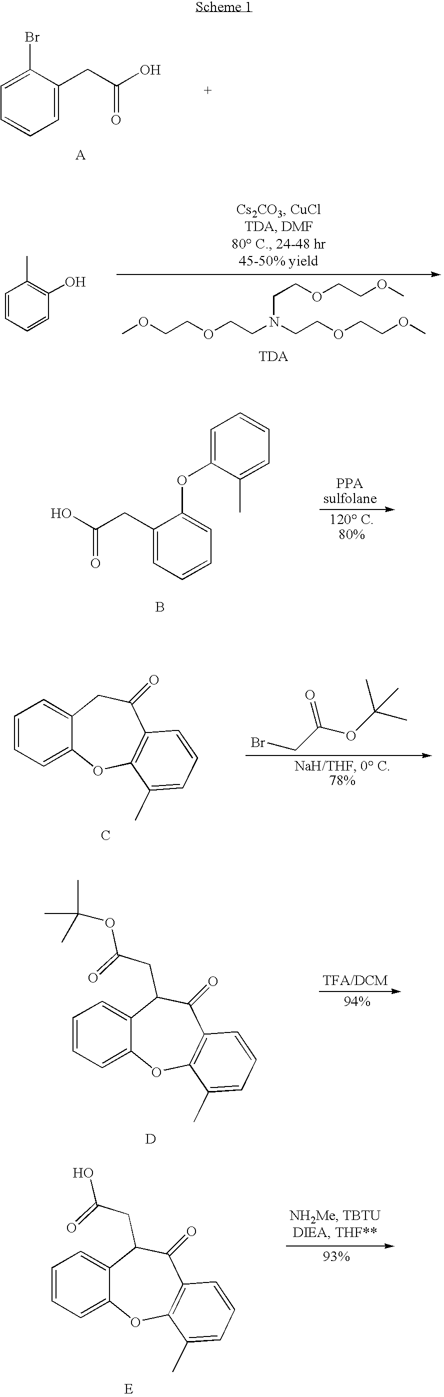

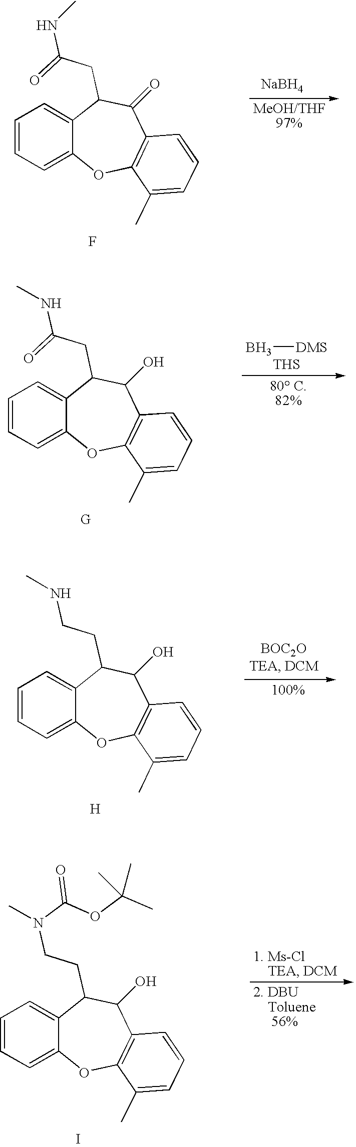

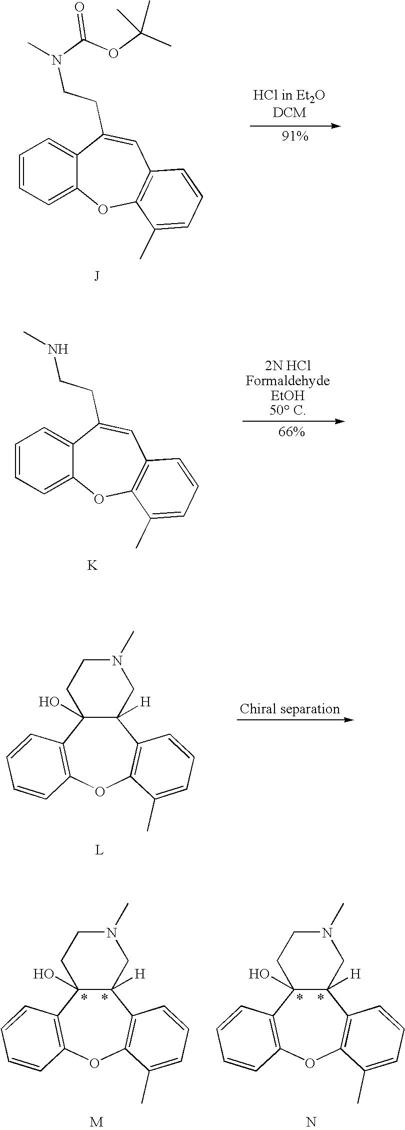

Specific synthetic details, as well as the conditions used for the chiral separation of the (+) and (−) beloxepin enantiomers are provided in the Examples section.

### 8.3 Activities and Uses of the Compounds and Compositions

As described in more detail in Example 2, (−)-beloxepin binds the norepinephrine (“NE”) transporter, and inhibits NE reuptake. The use of NRI compounds to treat a variety of diseases and disorders mediated, at least in part, by dysregulated NE reuptake is well documented. For example, the NRI atomoxetine (sold under the tradename STATTERA by Eli Lilly & Co.) is approved in the US for the treatment of attention deficit disorder (ADD) and attention deficit hyperactivity disorder (ADHD); the NRI reboxetine (sold under the tradename EDRONAX by Pharmacia-Upjohn) is approved in the UK and Ireland for the treatment of depressive illness; the NRI viloxazine (sold under the tradename VIVALAN by AstraZeneca) is approved in the US for the treatment of depression; the NRI maprotiline (sold under the tradename LUDIOMIL by Ciba-Geigy Corporation) is approved in the US for the treatment of depressive illness in patients with depressive neurosis (dysthymic disorder), manic-depressive illness, major depressive disorder and the relief of anxiety associated with depression; and the NRI nortriptyline (sold under the tradename Aventyl® by Eli Lilly) is approved in the US for the treatment of depressive disorders.

The ability of racemic (±)-beloxepin to cross the blood-brain barrier has been established in the literature (beloxepin has a reported logBB of 0.82; Kelder et al., 1999, Pharm. Res. 16:1514). Accordingly, the (−)-beloxepin compositions described herein are expected to be useful to treat any disease and/or disorder mediated, at least in part, by dysregulated NE reuptake. In some specific embodiments, it is expected that the (−)-beloxepin compositions described herein will be useful to treat all of the various diseases that respond to treatment with other NRI agents, including, by way of example and not limitation, atomoxetine, reboxetine, maprotiline, and nortriptyline. Diseases and disorders known to be mediated, at least in part, by dysregulated NE reuptake, and that are known to respond to treatment with NRI compounds, and that are expected to be treatable with the (−)-beloxepin compositions described herein, include, but are not limited to, urinary disorders, including urinary incontinence; mood disorders such as depression and seasonal affective disorder (SAD); cognitive disorders such as dementia; psychotic disorders such as schizophrenia and mania; anxiety disorders; personality disorders such as ADHD; eating disorders such as anorexia nervosa and bulimia nervosa; chemical dependencies resulting from addictions to drugs or substances of abuse such as addictions to nicotine, alcohol, cocaine, heroin, phenobarbital and benzodiazepines; withdrawal syndromes; endocrine disorders such as hyperprolactinaemia; impulse disorders such as trichotillomania and kleptomania; tic disorders such as Tourette's syndrome; gastrointestinal tract disorders such as irritable bowel syndrome (IBS), ileus, gastroparesis, peptic ulcer, gastroesophageal reflux disease (GORD, or its synonym GERD), flatulence and other functional bowel disorders such as dyspepsia (e.g., non-ulcerative dyspepsia (NUD)) and non-cardiac chest pain (NCCP); vascular disorders including vasospasms such as in the cerebral vasculature; and miscellaneous other disorders, including Parkinson's disease, shock and hypertension, sexual dysfunction, pre-menstrual syndrome and fibromyalgia syndrome.

One important class of diseases or disorders known to be responsive to treatment with NRIs is mental disease. Specific examples of such mental diseases or disorders include, but are not limited to, the various mental diseases and indications classified in the Diagnostic and Statistic Manual of Mental Disorders IV (Text Revision 2000; referred to hereinafter as “DSM-IV”) as mood disorders (such as, for example, depression), anxiety disorders (such as, for example OCD), eating disorders, (such as, for example, anorexia nervosa and bulimia nervosa), impulse disorders (such as, for example, trichotillomania), sleep disorders (such as, for example, insomnia related to opioid withdrawal), personality disorders (such as, for example, ADHD), and somatoform disorders (such as certain types of pain). In some embodiments, the (−)-compositions described herein are used to treat such mood disorders.

Pain is also thought to be mediated at least in part by NE reuptake. Pain is generally understood to refer to the perception or condition of unpleasant sensory or emotional experience, which may or may not be associated with actual damage to tissues. It is generally understood to include two broad categories, acute and chronic (see, e.g., Buschmann et al., (2002) “Analgesics,” Wiley VCH, Verlag GMbH & Co. KgaA, Weinheim; Jain, 2000, “Emerging Drugs” 5(2):241 257), and can be of either nociceptive origin (for example somatic or visceral) or non-nociceptive origin (for example neuropathic or sympathetic). Acute pain generally includes nociceptive pain arising from strains/sprains, burns, myocardial infarction, acute pancreatitis, surgery, trauma and cancer. Chronic pain generally includes nociceptive pain, including, but not limited to, inflammatory pain such as that associated with IBS or rheumatoid arthritis, pain associated with cancer and pain associated with osteoarthritis; and non-nociceptive pain, including, but not limited to, neuropathic pain (for example post-herpetic neuralgia, trigeminal neuralgia, focal peripheral nerve injury, anesthesia clolorosa), central pain (for example, post-stroke pain, pain due to spinal cord injury or pain associated with multiple sclerosis), and peripheral neuropathy (for example, diabetic neuropathy, inherited neuropathy or other acquired neuropathies).

Data presented in the Examples section confirms that (−)-beloxepin is effective at treating pain in a rodent model of neuropathic pain. Based upon this animal data, it is expected that the (−)-beloxepin compositions described herein will be useful in treating various different pain syndromes, including chronic pain of nociceptive origin, such as, for example, inflammatory pain, and chronic pain of non-nociceptive origin, such as, for example, neuropathic pain. Accordingly, in some embodiments, the (−)-beloxepin compositions described herein are used to treat pain, including the various types pain discussed above. It is also expected that the (−)-beloxepin compositions described herein will also be useful for blocking the onset of pain. In some embodiments, such compositions comprise beloxepin that is enriched in the (−) enantiomer. In some embodiments, such compositions comprise substantially enantiomerically pure (−)-beloxepin. In some embodiments, such compositions comprise enantiomerically pure (−)-beloxepin.

When used to treat various diseases or disorders discussed herein, the (−)-beloxepin composition will generally be administered in amounts effective to treat the particular disease or disorder. As will be recognized by skilled artisans, what is understood to be “therapeutically effective” and providing therapeutic benefit oftentimes depends upon the specific disease or disorder being treating. Skilled artisans will be able to ascertain a therapeutically effective amount based upon long established criteria for the particular indication.

In general, a “therapeutically effective” amount of a composition is an amount that eradicates or ameliorates the underlying disease or indication being treated and/or that eradicates or ameliorates one or more of the symptoms associated with the underlying disorder such that the patient reports an improvement in feeling or condition, not withstanding that the patient may still be afflicted with the underlying disease or indication. Therapeutic benefits also includes halting or slowing the progression of the disease or indication, regardless of whether improvement is realized.

In the context of depression, a therapeutically effective amount is an amount of composition that eradicates or ameliorates the depression or the symptoms thereof, including, but not limited to, changes in mood, feeling of intense sadness, despair, mental slowing, loss of concentration, pessimistic worry, agitation, self-deprecation, insomnia, anorexia, weight loss, decreased energy and libido, and hormonal circadian rhythms.

In the context of anxiety disorder, a therapeutically effective amount is an amount of composition that eradicates or ameliorates the anxiety disorder or one of the symptoms thereof including, but are not limited to, a fear of losing control of one's own actions, a sense of terror arising from no apparent reason, a dread of catastrophe, uneasiness, nervousness, nagging uncertainty about future events, headaches, fatigue, and sub-acute autonomic symptoms.

In the context of pain, a therapeutically effective amount is an amount of composition that eradicates or ameliorates the pain or the symptoms thereof, including, but not limited to, shooting sensations, burning sensations, electrical sensations, aching, discomfort, soreness, tightness, stiffness, sleeplessness, numbness, and weakness. An effective amount may also be an amount of a composition that blocks the onset of pain or the symptoms thereof. Thus, the composition may be administered therapeutically after the onset of sensation of pain or one or more of its symptoms, and/or prophylactically prior to the onset of sensation of pain or one or more of its symptoms. Is some embodiments, the composition may be administered in response to the sensation of pain or one or more of its symptoms and prophylactically thereafter to avoid its recurrence.

### 8.4 Combination Therapies

Compounds that inhibit NE reuptake have been used in combination with other therapies to treat various indications. For example, amitryptiline has been used in combination with chlordiazepoxide to treat anxiety disorder and major depressive disorder, and has been used in combination with perphenazine to treat anxiety disorder, schizophrenia and major depressive disorder. Nortryptiline has been used in combination with budenoside to treat asthma. It is expected that the (−)-beloxepin compositions described herein will also be useful in combination therapies.

When used in combination therapy, the (−)-beloxepin compositions described herein may be used in combination with, or as an adjunct to, other agents. When the (−)-beloxepin compositions described herein are used in combination with other agents, the two agents may be administered in a single pharmaceutical compositor or they may be administered in separate pharmaceutical compositions. The two components may be administered by the same route of administration or by a different route of administration. The two components also may be administered simultaneously with each other or sequentially. Thus each component of the combination therapy may be administered separately but sufficiently closely in time to the administration of the other component as to provide the desired effect.

While combination therapy involving the (−)-beloxepin compositions described herein is useful in many contexts, the other agent used with the (−)-beloxepin composition will depend on the specific disease or indication being treated. The skilled artisan will be able to ascertain what other agent to use in combination with the (−)-beloxepin compositions based upon long established criteria for the particular indication.

While not intending to be bound by any theory of operation, the combination therapy may include the administration of the (−)-beloxepin compositions described herein with other agents known to inhibit reuptake of NE. Alternatively, the combination therapy may include the administration of the (−)-beloxepin compositions with agents which do not inhibit the reuptake of NE. In some embodiments, the (−)-beloxepin compositions are administered in combination with compounds that inhibit other monoamine transporters, such as the 5HT transporter. In some specific embodiments, the (−)-beloxepin compositions are administered in combination with a selective serotonin reuptake inhibitor (SSRI), such as, but not limited to, fluoxetine, paroxetine, fluvoxamine, citaprolam, or sertraline, to treat depression. Combination therapy for the treatment of depression may also involve a monoamine oxidase inhibitor (MAOIs), such as, but not limited to, tranylcypromine, phenelzine, or isocarboxazid.

It is also expected that the (−)-beloxepin compositions described herein will be useful in combination therapy for the treatment of pain. Accordingly, the (−)-beloxepin compositions described herein can be combined with other analgesics, including but not limited to, cannabinoids and opioids. A number of cannabinoids are available that may be suitable for use in combination therapy, including, but not limited to, a cannabinoid that is selected from a Δ⁹-tetrahydrocannabinol and cannabidiol, and mixtures thereof.

Alternatively, the (−)-beloxepin compositions may be used in combination with at least one opioid. A wide variety of opioids are available that may be suitable for use in combination therapy to treat pain. As such, the combination therapy may involve an opioid that is selected from, but not limited to, alfentanil, allylprodine, alphaprodine, anileridine, benzyl-morphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, loperamide, meperidine (pethidine), meptazinol, metazocine, methadone, metopon, morphine, myrophine, nalbuphine, narceine, nicomorphine, norlevorphanol, normethadone, nalorphine, normorphine, norpinanone, opium, oxycodone, oxymorphone, papaveretum, pentazocine, phenadoxone, phenomorphan, phanazocine, phenoperidine, piminodine, piritramide, propheptazine, promedol, properidine, propiram, propoxyphene, sulfentanil, tilidine, tramadol, diastereoisomers thereof, pharmaceutically acceptable salts thereof, complexes thereof, and mixtures thereof. In some embodiments, the opioid is selected from morphine, codeine, oxycodone, hydrocodone, dihydrocodeine, propoxyphene, fentanyl, tramadol, and mixtures thereof.

The opioid component of the combination therapy may further include one or more other active ingredients that may be conventionally employed in analgesic and/or cough-cold-antitussive combination products. Such conventional ingredients include, for example, aspirin, acetaminophen, phenylpropanolamine, phenylephrine, chlorpheniramine, caffeine, and/or guaifenesin. Typical or conventional ingredients that may be included in the opioid component are described, for example, in the Physicians' Desk Reference, 1999, the disclosure of which is hereby incorporated herein by reference, in its entirety.

The opioid component may further include one or more compounds that may be designed to enhance the analgesic potency of the opioid and/or to reduce analgesic tolerance development. Such compounds include, for example, dextromethorphan or other NMDA antagonists (Mao et al., 1996, Pain 67:361), L-364,718 and other CCK antagonists (Dourish et al., 1988, Eur. J. Pharmacol 147:469), NOS inhibitors (Bhargava et al., 1996, Neuropeptides 30:2), PKC inhibitors (Bilsky et al., 1996, J. Pharmacol. Exp. Ther. 277:484), and dynorphin antagonists or antisera (Nichols et al., 1997, Pain 69:317). The disclosures of each of the foregoing documents are hereby incorporated herein by reference, in their entireties.

Alternatively, the (−)-beloxepin compositions may be used with at least one non opioid analgesic, such as for example, diclofenac, a COX2 inhibitor, aspirin, acetaminophen, ibuprofen, naproxen, and the like, and mixtures thereof.

Other agents that may be used in combination with the (−)-beloxepin compositions described herein include anti-inflammatory agents, including but not limited to non-steroidal anti-inflammatory drugs (“NSAIDs”). Specific examples of suitable anti-inflammatory agents include, but are not limited to, corticosteroids, aminoarylcarboxylic acid derivatives such as, but not limited to, etofenamate, meclofenamic acid, mefanamic acid, niflumic acid; arylacetic acid derivatives such as, but not limited to, acemetacin, amfenac cinmetacin, clopirac, diclofenac, fenclofenac, fenclorac, fenclozic acid, fentiazac, glucametacin, isozepac, lonazolac, metiazinic acid, oxametacine, proglumetacin, sulindac, tiaramide and tolmetin; arylbutyric acid derivatives such as, but not limited to, butibufen and fenbufen; arylcarboxylic acids such as, but not limited to, clidanac, ketorolac and tinoridine; arylpropionic acid derivatives such as, but not limited to, bucloxic acid, carprofen, fenoprofen, flunoxaprofen, ibuprofen, ibuproxam, oxaprozin, piketoprofen, pirprofen, pranoprofen, protizinic acid and tiaprofenic add; pyrazoles such as, but not limited to, mepirizole; pyrazolones such as, but not limited to, clofezone, feprazone, mofebutazone, oxyphenbutazone, phenylbutazone, phenyl pyrazolidininones, suxibuzone and thiazolinobutazone; salicylic acid derivatives such as, but not limited to, bromosaligenin, fendosal, glycol salicylate, mesalamine, 1-naphthyl salicylate, olsalazine and sulfasalazine; thiazinecarboxamides such as, but not limited to, droxicam, isoxicam and piroxicam; and other anti-inflammatory agents such as, but not limited to, e-acetamidocaproic acid, S-adenosylmethionine, 3-amino-4-hydroxybutyric acid, amixetrine, bendazac, bucolome, carbazones, difenpiramide, ditazol, guaiazulene, heterocyclic aminoalkyl esters of mycophenolic acid and derivatives, nabumetone, nimesulide, orgotein, oxaceprol, oxazole derivatives, paranyline, pifoxime, 2-substituted-4,6-di-tertiary-butyl-s-hydroxy-1,3-pyrimidines, proquazone and tenidap.

### 8.5 Advantages of (−)-Beloxepin Combination Therapy

As evidenced by Example 4, (−)-beloxepin does not appreciably inhibit the polymorphic cytochrome P450 isoenzyme CYP2D6 (IC₅₀=4370 nM). Many drugs that would be useful in compositions described herein are metabolized or activated by CYP2D6. Since (−)-beloxepin does not appreciably inhibit this P450 isoenzyme, combination therapy with (−)-beloxepin can be applied without having to alter dosages of drugs metabolized by or activated by CPY2D6.

Cytochrome P4502D6 (CYP2D6) is a polymorphic member of the P450 superfamily, which is absent in 5-9% of the Caucasian population, resulting in a deficiency in drug oxidation known as debrisoquine/sparteine polymorphism. Metabolism by polymorphic isoenzymes such as CYP2D6 can be problematic in drug development because of the wide variation in the pharmacokinetics of the patient population. CYP2D6 metabolises many currently used drugs, which include β-blockers, antidepressants, and neuroleptics (Bertz and Granneman, (1997) Clin. Pharmokinet. 32(3):210-58). Polymorphisms of 2D6 have been associated with a reduced capacity to dispose important drugs; this leads to undesirable clinical consequences (Ingelman-Sundberg et al. (1999) Trends Pharmacol. Sci. 20(8):342-49). The impact of human P450 polymorphisms on drug treatment in poor metabolizers is indicated in Table 2, below (see (Ingelman-Sundberg et al. (1999) Trends Pharmacol. Sci. 20(8):342-49):

TABLE 2
Impact of human P450 polymorphisms on
drug treatment in poor metabolizers

| Polymorphic | | | Reduced prodrug |
|---|---|---|---|
| enzyme | Decreased clearance | Adverse effects | activation |
| CYP 2C9 | S-Warfarin | Bleeding | Losartan |
| | PHenytoin | Ataxia | |
| | Losartan | | |
| | Tolbutamide | Hypoglycaemia | |
| | NSAIDs | GI bleeding | |
| CYP 2C19 | Omeprazole | | Proguanil |
| | Diazepam | Sedation | |
| CP2D6 | Tricyclic | Cardiotoxicity | Tramadol |
| | antidepressants | | Codeine |
| | Haloperidol | Parkinsonism | Ethylmorphine |
| | Anti-arrhythmic drugs | Arrhythmias | |
| | Perphenazine | | |
| | Perhexiline | Neuropathy | |
| | SSRIs | Nausea | |
| | Zuclopenthixol | | |
| | S-Mianserin | | |
| | Tolterodine | | |

Abbreviations: NSAIDs, nonsteroidal anti-inflammatory drugs; SSRIs, selective serotonin reuptake inhibitors

As indicated in Table 2, inhibition of the metabolizing activity of polymorphic P450 enzymes can have serious clinical consequences. Such inhibitory activity must be taken into account when two or more therapeutic agents are to be administered in combination to a patient, where one may affect the P450 mediated-clearance of the other.

### 8.6 Formulations and Administration

The (−)-beloxepin compound or pharmaceutical salts thereof may be combined with a pharmaceutical carrier selected on the basis of the chosen route of administration and standard pharmaceutical practice as described, for example, in Remington 's Pharmaceutical Sciences, 2005, the disclosure of which is hereby incorporated herein by reference, in its entirety. The relative proportions of active ingredient and carrier may be determined, for example, by the solubility and chemical nature of the compounds, chosen route of administration and standard pharmaceutical practice.

The (−)-beloxepin compound and/or compositions described herein may be administered to a mammalian subject in a variety of forms adapted to the chosen route of administration, e.g., orally or parenterally. Parenteral administration in this respect includes administration by the following routes: intravenous, intramuscular, subcutaneous, intraocular, intrasynovial, transepithelial including transdermal, ophthalmic, sublingual and buccal; topically including ophthalmic, dermal, ocular, rectal and nasal inhalation via insufflation, aerosol and rectal systemic.

The (−)-beloxepin compound and/or compositions may be formulated for oral administration, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is preferably such that a suitable dosage will be obtained. Preferred compositions or preparations may be prepared so that an oral dosage unit form contains from about 0.1 to about 1000 mg of each active compound (and all combinations and subcombinations of ranges and specific concentrations therein).

The tablets, troches, pills, capsules and the like may also contain one or more of the following: a binder such as gum tragacanth, acacia, corn starch or gelatin; an excipient, such as dicalcium phosphate; a disintegrating agent such as corn starch, potato starch, alginic acid and the like; a lubricant such as magnesium stearate; a sweetening agent such as sucrose, lactose or saccharin; or a flavoring agent such as peppermint, oil of wintergreen or cherry flavoring. When the dosage unit form is a capsule, it may contain, in addition to materials of the above type, a liquid carrier. Various other materials may be present as coating, for instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non toxic in the amounts employed.

The (−)-beloxepin compound and/or compositions may also be formulated for parental or intraperitoneal administration. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols, and mixtures thereof and in oils. Under ordinary conditions of storage and use, these preparations may contain a preservative to prevent the growth of microorganisms.

Compositions suitable for administration by injection typically include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze drying technique that yields a powder of the active ingredient, plus any additional desired ingredient from the previously sterile filtered solution thereof.

### 8.7 Effective Dosages

The (−)-beloxepin compound and/or compositions will generally be administered in a therapeutically effective amount, as described herein. The amount of compound or composition administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, etc. Determination of an effective dosage is well within the capabilities of those skilled in the art.

Dosage amounts will typically be in the range of from about 0.0001 or 0.001 or 0.01 mg/kg/day to about 0.1 or 1.0 or 2.0 or 2.5 or 5.0 or 10.0 or 20.0 or 25.0 or 50.0 or 75.0 or 100 mg/kg/day with an expected dose of about 5 mg/day to about 1500 mg/day, but may be higher or lower, depending upon, among other factors, the particular disease or indication being treated the activity of the compound and/or composition, its bioavailability, the mode of administration and various factors discussed above. Dosage amount and interval may be adjusted individually to provide plasma levels of the compounds and/or compositions which are sufficient to maintain therapeutic or prophylactic effect. As non-limiting examples, the compounds and/or compositions may be administered once per day or multiple times per day, depending upon, among other things, the mode of administration, the specific indication being treated and the judgment of the prescribing physician. In cases of local administration or selective uptake, such as local topical administration, the effective local concentration of active compounds and/or compositions may not be related to plasma concentration. Skilled artisans will be able to optimize effective local dosages without undue experimentation.

Initial dosages of the (−)-beloxepin compound and/or compositions useful for the treatment of pain can be estimated from in vivo data, such as the animal data described in the Examples section.

Based on the animal data described in Examples 3, 6, and 7, it is expected that an effective dosage of (−)-beloxepin for the treatment of pain in humans may be obtained by administering a dose of (−)-beloxepin sufficient to achieve a plasma concentration similar to that achieved following the administration of 30 mg/kg, i.p. to rats. As such, in some embodiments the effective dose of (−)-beloxepin for the treatment of pain is the dosage required to achieve the plasma concentration achieved when 30 mg/kg (−)-beloxepin is administered i.p. to rats.

Based on these animal data, it is expected that oral doses of (−)-beloxepin of between about 10 mg/day to about 20 or 25 or 30 or 35 or 40 or 45 or 50 or 60 or 70 or 80 or 90 or 95 or 100 or 200 or 500 or 750 or 1000 or 1250 or 1500 mg/day (including any incremental amount between 10 and 1500 mg/day) will be effective in treating pain. Accordingly, some embodiments involve the administration of an oral dosage of (−)-beloxepin that ranges from about 5 mg/day to about 500 mg, one or more times per day. In some embodiments, a patient is administered 500 mg (−)-beloxepin composition orally twice per day.

In the context of combination therapy, the proper dosage of the combined agents will be readily ascertainable by a skilled artisan based on the above disclosed dosages for (−)-beloxepin and long established criteria for the particular indication. By way of general guidance, where a cannabinoid, opioid and/or other agent is used in combination with the (−)-beloxepin compositions described herein, the dosage will typically range from about 0.01 to about 100 mg/kg/day of the cannabinoid, opioid and/or other active compound and about 0.001 to about 100 mg/kg/day of (−)-beloxepin. In certain embodiments, the dosage may be about 0.1 to about 10 mg/kg/day of the cannabinoid, opioid and/or other active compound and about 0.01 to about 10 mg/kg/day of (−)-beloxepin, and in other embodiments, the daily dosage may be about 1.0 mg of the cannabinoid, opioid and/or other active compound and about 0.1 mg of (−)-beloxepin. Alternatively, when the (−)-beloxepin compositions described herein are combined with a cannabinoid compound (e.g., Δ⁹-tetrahydrocannabinol or cannabidiol), an opioid compound (e.g., morphine) and/or an other agent and the combination is administered orally, the dosage may generally range from about 15 to about 200 mg of the cannabinoid, opioid and/or other agent, and about 0.1 to about 4 mg of (−)-beloxepin.

### 8.8 Kits

The (−)-beloxepin compounds and/or pharmaceutical salts thereof described herein may be assembled in the form of kits. In some embodiments, the kit provides the compounds(s) and reagents to prepare a composition for administration. The composition may be in a dry or lyophilized from, or in a solution, particularly a sterile solution. When the composition is in a dry form, the reagent may comprise a pharmaceutically acceptable diluent for preparing a liquid formulation. The kit may contain a device for administration or for dispensing the compositions, including, but not limited to, syringe, pipette, transdermal patch or inhalant.

The kits may include other therapeutic agents for use in conjunction with the compounds described herein. In some embodiments, the therapeutic agents may be provided in a separate form, or mixed with the compounds described herein.

Kits can include appropriate instructions for preparation and administration of the composition, side effects of the compositions, and any other relevant information. The instructions may be in any suitable format; including, but not limited to, printed matter, videotape, computer readable disk, or optical disk.

### 9. EXAMPLES

The following working examples, which are intended to be illustrative and not limiting, highlight various features of certain embodiments of (−)-beloxepin compositions and methods described herein.

### Example 1

### Synthesis of (±)-Beloxepin and Isolation of (−)-Beloxepin

With reference to Scheme 1, reproduced below, enantiomerically pure (−)-beloxepin was obtained as follows.

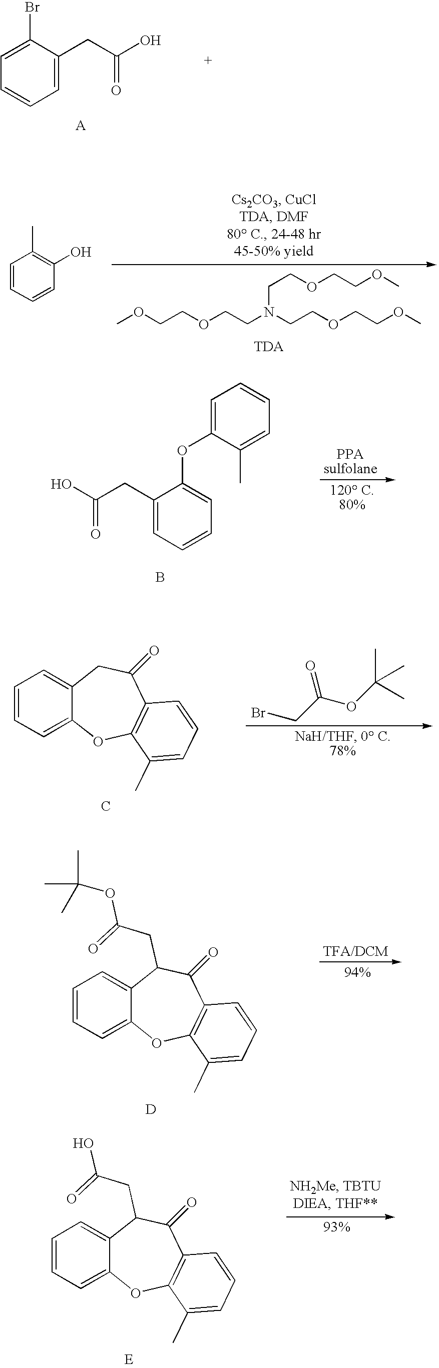

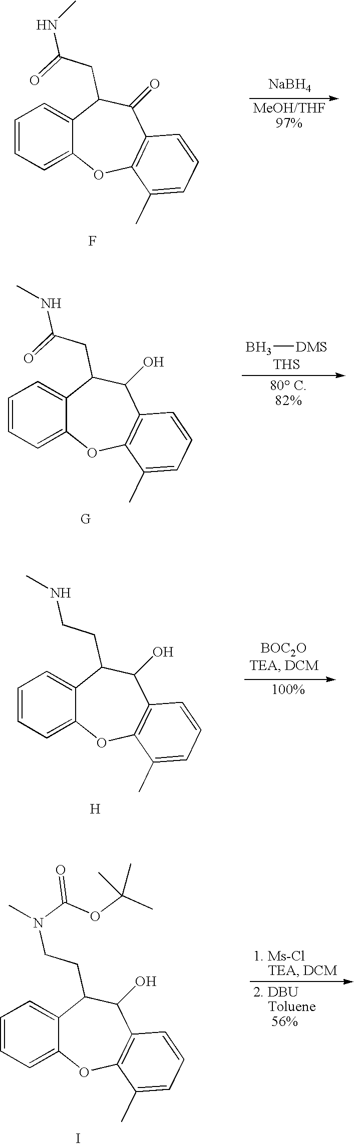

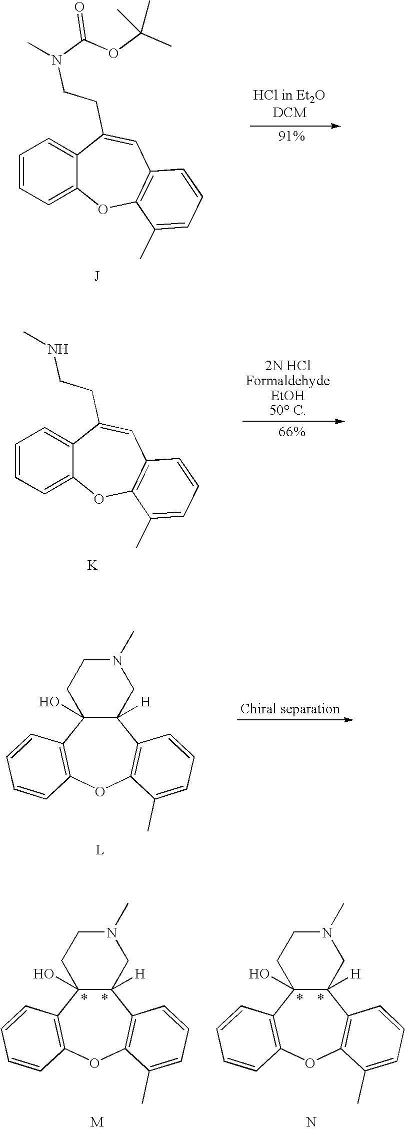

Preparation of 2-(2-(o-tolyloxy)phenyl)acetic acid (B): To a solution of A (50.0 g, 232 mmol, 1.00 eq) in N,N-dimethylformamide (500 mL) under nitrogen and with mechanical stirring was added cesium carbonate (189 g, 581 mmol, 2.50 eq), o-cresol (28.8 mL, 279 mmol, 1.20 eq), copper(I) chloride (12 g, 120 mmol, 0.5 eq) and tris(3,6-dioxaheptyl)amine (TDA) (37 mL, 120 mmol, 0.5 eq). The reaction was degassed by bubbling nitrogen through the stirring mixture for 10 minutes. The mixture was then heated at 80° C. for 2 days under nitrogen. The reaction was cooled to room temperature and diluted with 1:1 diethyl ether/hexanes. While stirring, the mixture was carefully acidified with 6M HCl, then diluted with water and the layers were separated. The aqueous layer was washed with 1:1 diethyl ether/hexanes and all organics were combined and washed with 0.5M sodium carbonate. The basic aqueous layers were combined, acidified with 6M HCl and the product was extracted with diethyl ether. The organics were concentrated and purified by a silica gel plug using 2-5% isopropanol/hexane gradient to give 31.48 g yellow/green oil (51% yield, based on ¹H NMR purity of 92%). ¹H NMR (400 MHz, CDCl₃) 7.29 (dd, 1H), 7.23-7.10 (m, 3H), 7.05 (m, 2H), 6.83 (dd, 1H), 6.63 (dd, 1H), 3.77 (s, 2H), 2.20 (s, 3H); MS: (M−H)⁻=241.1.

Preparation of 6-methyldibenzo[b,f]oxepin-10(11H)-one (C): A mixture of B (60.7 g, 213 mmol, 1.00 eq, 85% purity), polyphosphoric acid (93 g, 852 mmol, 4.00 eq) and sulfolane (200 mL) was immersed in an oil bath at 120° C. and heated for 90 minutes. Ice water was added and the product was extracted with diethyl ether. The organic layer was washed with 0.5 M sodium carbonate, concentrated and purified by a silica gel plug using a 1-4% ethyl acetate/hexanes gradient to give 41.4 g orange oil (80%**). **Yield based on 85% purity of starting material B and 92% purity of product C. ¹H NMR (400 MHz, CDCl₃) 7.91 (m, 1H), 7.44 (m, 1H), 7.32 (m, 1H), 7.25 (m, 2H), 7.19 (m, 1H), 7.07 (m, 1H), 4.10 (s, 2H), 2.57 (s, 3H)

Preparation of (4-Methyl-11-oxo-10,11-dihydro-dibenzo[b,f]oxepin-10-yl)-acetic acid tert-butyl ester (D): To a mixture of 60% sodium hydride in mineral oil (8.16 g, 204 mmol, 1.2 eq) in tetrahydrofuran (400 mL) cooled in a brine/water bath was added dropwise a solution of the ketone C (41.4 g, 170 mmol, 1.0 eq, 92% purity) in tetrahydrofuran (200 mL). The mixture was stirred for an additional 10 minutes. The bromide was added dropwise over a 10 minutes period and the reaction was stirred cooled for 40 minutes. The reaction was quenched with water and concentrated. The crude product was partitioned between water and diethyl ether, layers were separated and the organics were washed with brine. The organics were concentrated and the resulting solid was triturated in hexanes, filtered and dried to give 44.1 g of an off-white solid. The filtrate was concentrated and there were crystals after 3 days. Crystals were filtered and dried to give 1.5 g pale orange crystalline solid. Total yield=78%. ¹H NMR (400 MHz, CDCl₃) 7.86 (dd, 1H), 7.43 (m, 1H), 7.25-7.20 (m, 4H), 7.06 (t, 1H), 4.83 (m, 1H), 3.37 (m, 1H), 2.87 (dd, 1H), 2.57 (s, 3H), 1.42 (s, 9H); MS: M⁺=338.4

Preparation of (4-Methyl-11-oxo-10,11-dihydro-dibenzo[b,f]oxepin-10-yl)-acetic acid (E): The ester D (44.0 g, 128 mmol, 1.0 eq) was dissolved in dichloromethane (500 mL) and trifluoroacetic acid (34.5 mL, 448 mmol, 3.5 eq) was added. The reaction was stirred at room temperature over 48h. The reaction was diluted with water and the layers were separated. The organics were concentrated, triturated in 1:1 diethyl ether/hexanes (250 mL), filtered and dried to give 34.6 g of a pale yellow solid (94%). ¹H NMR (400 MHz, DMSO) 12.40 (brs, 1H), 7.72 (dd, 1H), 7.61 (m, 1H), 7.44 (m, 1H), 7.36-7.30 (m, 3H), 7.18 (t, 1H), 4.73 (m, 1H), 3.33 (m, 1H), 2.92 (dd, 1H), 2.57 (s, 3H); MS: (M−H)⁻=281.2

Preparation of N-Methyl-2-(4-methyl-11-oxo-10,11-dihydro-dibenzo[b,f]oxepin-10-yl)-acetamide (F): The acid E (34.5 g, 120 mmol, 1.0 eq) was suspended in tetrahydrofuran (200 mL) under nitrogen. To the mixture was added N,N-diisopropylethylamine (31.3 mL, 180 mmol, 1.5 eq), methyl amine (120 mL, 240 mmol, 2.0 eq) and TBTU (46.2 g, 144 mmol, 1.2 eq). The reaction was stirred at room temperature for 2 hours. Between 30 and 60 minutes, a thick precipitate forms and the reaction turns light green. Another 100 mL of tetrahydrofuran was added and slow stirring resumed. N,N-dimethylformamide (100 mL) was added followed by additional amount of TBTU (15 g). The reaction mixture was concentrated to near dryness and the product was partitioned between diethyl ether and a 50% aqueous solution of sodium bicarbonate. The aqueous was washed with diethyl ether and all organics were combined and concentrated. The resulting solid was triturated in 300 mL 1:1 diethyl ether/hexanes, filtered and dried to give 33.3 g off-white solid (93%). ¹H NMR (400 MHz, CDCl₃) 7.84 (dd, 1H), 7.43 (m, 1H), 7.25-7.20 (m, 3H), 7.16 (m, 1H), 7.06 (t, 1H), 4.96 (dd, 1H), 3.33 (m, 1H), 2.82 (d, 3H), 2.75 (dd, 1H), 2.57 (s, 3H); MS: (M+H)⁺=296.0

Preparation of 2-(11-Hydroxy-4-methyl-10,11-dihydro-dibenzo[b,f]oxepin-10-yl)-N-methyl-acetamide (G): The ketone F (33.2 g, 112 mmol, 1.0 eq) was partially dissolved in methanol/tetrahydrofuran (200 mL/200 mL) under nitrogen and cooled in an ice/water bath. Sodium borohydride (10.6 g, 281 mmol, 2.5 eq) was added in 2 g portions over a 15 minutes period. The ice bath was removed and the mixture was stirred at room temperature for 1 hour. The reaction was quenched with water and concentrated to near dryness. The crude product was suspended in dichloromethane, water was added and the layers were separated. The aqueous layer was washed again with dichloromethane and the organics were combined and concentrated. To the resulting foam was added 250 mL of 1:1 diethyl ether/hexanes with vigorous stirring. A white precipitate immediately formed and it was filtered and dried to give 32 g of a white powder (97%); MS: (M+H)⁺=298.0

Preparation of 6-Methyl-1′-(2-methylamino-ethyl)-10,11-dihydro-dibenzo[b,f]oxepin-10-ol (H): The amide G (31.9 g, 107 mmol, 1.0 eq) was dissolved in tetrahydrofuran (200 mL) under nitrogen and the borane-dimethyl sulfide complex (2.0 M in tetrahydrofuran, 161 mL, 322 mmol, 3.0 eq) was added dropwise over 15 minutes. The reaction was then heated at 80° C. for 24 hours. The reaction was cooled in an ice/water bath and methanol (50 mL) was added in 10 mL portions over 30 minutes. The mixture was stirred for 30 minutes at room temperature. A solution of 4M HCl in dioxane (130 mL, ˜5 eq) was added dropwise over 15 minutes. The mixture was stirred at room temperature for 30 minutes. The mixture was concentrated to near dryness and water and 10% ethyl acetate/diethyl ether were added. Layers were separated and the aqueous phase was washed with 10% ethyl acetate/diethyl ether. The aqueous layer was basified with a saturated sodium bicarbonate solution and the product was extracted with 10% methanol/dichloromethane. The organics were combined, dried over sodium sulfate, concentrated and dried to give 25.8 g of a yellow oil (82%). MS: (M+H)⁺=284.0

Preparation of [2-(11-hydroxy-4-methyl-10,11-dihydro-dibenzo[b,f]oxepin-10-yl)-ethyl]-methyl-carbamic acid tert-butyl ester (I): To a solution of the amine H (25.0 g, 86 mmol, 1.0 eq, 96.9% pure) and triethylamine (14.3 mL, 102 mmol, 1.2 eq) in dichloromethane (300 mL) was added di-tert-butyldicarbonate (19.6 g, 90 mmol, 1.05 eq) portion wise. The reaction was stirred at room temperature for 15 minutes. The reaction was diluted with 0.5 M HCl and the layers were separated. The organics were washed with 0.5 M HCl, dried over sodium sulfate, concentrated and dried to give 35 g of a yellow oil (100% yield based on 93% purity).

MS: (M+H)⁺=384.0

Preparation of methyl-[2-(4-methyl-dibenzo[b,f]oxepin-10-yl)-ethyl]-carbamic acid tert-butyl ester (J): The alcohol I (23.5 g, 57 mmol, 1.0 eq, 93% purity) was dissolved in dichloromethane (300 mL) and triethylamine (20.6 mL, 148 mmol, 2.6 eq) was added. The mixture was cooled in an ice bath and methanesulfonyl chloride (5.73 mL, 74 mmol, 1.3 eq) was added. The reaction mixture was stirred cooled for 15 minutes. The reaction mixture was diluted with 0.5 M HCl and the layers were separated. The organics were concentrated and dried to give 28 g of a crude light yellow oil. The mesylate was dissolved in toluene (200 mL) and 1,8-diazabicyclo[5.4.0]undec-7-ene (42.6 mL, 285 mmol, 5.0 eq) was added. The mixture was heated at 115° C. for 1 hour and diluted with water. The layers were separated and the organics were concentrated and purified by a silica gel plug eluting with 5-15% ethyl acetate/hexanes to give 14.76 g of a light yellow oil. This total amount was collected in two batches (8.44 g, 81% pure by LC/MS) and (6.32 g, 77% pure by LC/MS). ¹H NMR (400 MHz, CDCl₃) 7.40 (brm, 1H), 7.28 (m, 1H), 7.22-7.10 (m, 3H), 6.98 (m, 2H), 6.70 (brs, 1H), 3.39 (brm, 2H), 2.91-2.82 (brm, 5H), 2.53 (s, 3H), 1.46 (s, 9H); MS: (M+H)⁺⁼366.0

Preparation of methyl-[2-(4-methyl-dibenzo[b,f]oxepin-10-yl)-ethyl]-amine (K): The olefin J (14.8 g, 32 mmol, 1.0 eq, 79% pure) was dissolved in dichloromethane (150 mL) and a solution of HCl in diethyl ether (2.0M, 75 mL, 160 mmol, 5 eq) was added. The mixture was stirred overnight at room temperature. The reaction was diluted with a solution of saturated sodium bicarbonate and layers were separated. The aqueous layer was washed with 10% methanol/dichloromethane and all organics were combined, concentrated and purified by a flash silica gel column using a 2-10% methanol/dichloromethane gradient (plus 1% NH₄OH) to give 8.0 g of a yellow oil in 91% yield and 96% purity. ¹H NMR (400 MHz, CDCl₃) 7.38 (m, 1H), 7.30 (m, 2H), 7.15 (m, 2H), 6.99 (m, 2H), 6.74 (s, 1H), 2.93 (t, 2H), 2.78 (t, 2H), 2.52 (s, 3H), 2.44 (s, 3H); MS: (M+H)⁺=266.0

Preparation of L: To the amine K (7.0 g, 25 mmol, 1.0 eq) under nitrogen was added ethanol (23 mL), an aqueous solution of HCl (2.0 M, 226 mL, 19 eq) and an aqueous solution of formaldehyde (37%, 100 mL, 52 eq). The reaction mixture was heated at 50° C. for 64 hours. The reaction mixture was cooled in an ice bath and it was basified with 2M NaOH to pH ˜8. The product was extracted with 10% methanol/dichloromethane. The organics were combined, concentrated and purified by a flash silica gel column using a 4-9% methanol/dichloromethane gradient (plus 1% NH₄OH) to give 4.9 g white solid in 66% yield and 100% purity. ¹H NMR (400 MHz, CDCl₃) 7.62 (d, 1H), 7.27 (m, 3H), 7.14 (m, 1H), 7.08 (m, 1H), 7.00 (m, 1H), 3.28 (brs, 1H), 3.10 (brt, 1H), 3.00 (brm, 1H), 2.82 (brm, 1H), 2.46 (brs, 1H), 2.42 (s, 3H), 2.29 (s, 3H), 2.18 (m, 1H), 2.03 (s, 1H), 1.80 (brm, 1H); MS: (M+H)⁺=296.0. CHN Theory (1 mol H₂O): % C 72.82% H 7.40% N 4.47. CHN Actual (1 mol H₂O): % C 72.69% H 7.29% N 4.48

Preparation of M and N: the Chiral Separation of the Racemic Mixture L (Racemic beloxepin) was conducted using the following conditions: (i) Column: Chiralpak AD-H, 21×250 mm, 5 micron; (ii) Flow: 15 mL/min, (iii) Mobile phase: 60% Methanol (0.2% triethylamine), 20% ethanol, 20% hexane; and (iv) Detection: 270 nm.

M: Peak Retention Time: Peak 2 [(−)-beloxepin]=5.8 min. [α]_{D}23.7=−111.34 (c. 12.0 mg/mL, MeOH). ¹H NMR (400 MHz, CDCl₃) 7.62 (d, 1H), 7.27 (m, 3H), 7.14 (m, 1H), 7.08 (m, 1H), 7.00 (m, 1H), 3.27 (brm, 1H), 3.08 (t, 1H), 2.98 (m, 1H), 2.79 (brm, 1H), 2.46 (brs, 1H), 2.41 (s, 3H), 2.27 (s, 3H), 2.15 (m, 1H), 2.07 (brs, 1H), 1.85 (brm, 1H); MS: (M+H)⁺=296.0; CHN Theory: % C 77.26% H 7.17% N 4.74 and CHN Actual: % C 77.16% H 7.25% N4.76

N: Peak Retention Time: Peak 1 [(+)-beloxepin]=4.7 min. [α]_{D}23.7=+110.80 (c. 11.1 mg/mL, MeOH); ¹H NMR (400 MHz, CDCl₃) 7.62 (d, 1H), 7.27 (m, 3H), 7.15 (m, 1H), 7.08 (m, 1H), 7.00 (m, 1H), 3.27 (brm, 1H), 3.08 (t, 1H), 2.98 (m, 1H), 2.80 (brm, 1H), 2.46 (brs, 1H), 2.42 (s, 3H), 2.28 (s, 3H), 2.15 (m, 1H), 2.05 (s, 1H), 1.80 (brm, 1H); MS: (M+H)⁺=296.0; CHN Theory: % C 77.26% H 7.17% N 4.74 and CHN Actual: % C 76.96% H 7.24% N4.74

### Example 2

### (−)-Beloxepin Selectively Binds the Norepinephrine Transporter and Inhibits Reuptake of Norepinephrine

The binding affinities of (±)-, (−)- and (+)-beloxepin for the NE and serotonin transporters and the 5HT_{2A}, 5HT_{2B} and 5HT_{2C} receptors were determined in competitive binding assays with radiolabeled ligands. The ability of these compounds to inhibit reuptake of NE and 5HT, as well as the ability to agonize and antagonize the 5HT_{2A}, 5HT_{2B} and 5HT_{2C} receptors was also studied. Beloxepin had only marginal affinity at the serotonin and dopamine transporters (SERT: 27% inhibition at 10 μM; DAT: 16% inhibition at 10 μM).

Protocols. For the NE transporter binding assay, [³H]nisoxetine (1.0 nM) was incubated with various concentrations of test compounds for 2 hours at 4° C. with membranes prepared from Chinese hamster ovary cells (CHO) cells heterologously expressing the cloned human NE transporter (hNET). Bound radioactivity was determined by scintillation spectroscopy. Non-specific binding was defined as the amount of binding that occurred in the presence of 1.0 μM desipramine. The Kᵢ values of the various test compounds were determined using standard methods.

For the NE uptake inhibition assay, IC₅₀ values were determined by measuring the inhibition of the incorporation of [³H]norepinephrine into rat hypothalamus synaptosomes upon incubation for 20 minutes at 37° C.

For the 5HT transporter binding assay, [³H] imipramine (2.0 nM) was incubated in the presence of various concentrations of test compounds for 1 hour at 22° C. with membranes prepared from CHO cells heterologously expressing the human serotonin transporter (hSERT). Bound radioactivity was determined by scintillation spectroscopy. Non-specific binding was defined as the amount of binding that occurred in the presence of 10 μM imipramine. The Kᵢ values of the various test compounds were determined using standard methods.

For the 5HT uptake inhibition assay, IC₅₀ values were determined by measuring the inhibition of the incorporation of [³H]-5HT into rat brain synaptosomes upon incubation for 15 min at 37° C.

For the 5HT_{2A} receptor binding assay, [³H]ketanserin (0.5 nM) was incubated for 60 min at 22° C. with membranes prepared from HEK-293 cells heterologously expressing the cloned human 5-HT_{2A} receptor according to the method of Bonhaus et al., 1995, Brit. J. Pharmacol. 115:622-628. Various concentrations of test compound were added and bound radioactivity was determined by scintillation counting. Non-specific binding was determined in the presence of 1.0 μM unlabeled ketanserin. The Kᵢ value for the test compound was determined using standard methods.

For the 5HT_{2B} receptor binding assay, [¹²⁵I](±)1,2,5-dimethoxy-4,2-aminopropane (DOI) (0.2 nM) was incubated for 15 min at 37° C. with membranes prepared from Chinese hamster ovary cells heterologously expressing the cloned human 5HT_{2B} receptor according to the method of Choi et al., 1994, FEBS Lett 352:393-399. Various concentrations of test compound were added and bound radioactivity was determined by scintillation counting. Non-specific binding was determined in the presence of 1.0 μM unlabeled DOI. The Kᵢ value for the test compound was determined using standard methods.

For the 5HT_{2C} receptor binding assay, [³H]mesulergine (1.0 nM) was incubated for 60 min at 37° C. with membranes prepared from Chinese hamster ovary cells heterologously expressing the cloned human 5-HT_{2C} receptor according to the method of Stam et al., 1994, Eur. J. Pharmacol. 269:339-348. Various concentrations of test compound were added and bound radioactivity was determined by scintillation counting. Non-specific binding was determined in the presence of 10 μM RS102221. The Kᵢ value for the test compound was determined using standard methods.

Agonist effects at the 5HT_{2A} receptor were assessed by incubation at 22° C. of a series of concentrations of test compound with intact HEK-293 cells heterologously expressing the cloned human 5HT_{2A} receptor and measuring intracellular [Ca²⁺] by fluorimetry according to the method of Jerman et al., 2001, Eur. J. Pharmacol. 414:23-30). Antagonist effects were assessed by the ability of a series of concentrations of test compound to block the increase in intracellular [Ca²⁺] that occurred in the presence of 3.0 nM serotonin under the same conditions. EC₅₀ and IC₅₀ values were determined using standard methods.

Agonist effects at the 5HT_{2B} receptor were assessed by incubation at 22° C. of a series of concentrations of test compound with intact CHO cells heterologously expressing the cloned human 5HT_{2B} receptor and measuring intracellular [Ca²⁺] by fluorimetry according to the method of Porter et al., 1991, Brit. J. Pharmacol. 128:13-20. Antagonist effects were assessed by the ability of a series of concentrations of test compound to block the increase in intracellular [Ca²⁺] that occurred in the presence of 0.3 nM serotonin under the same conditions. EC₅₀ and IC₅₀ values were determined using standard methods.

Agonist effects at the 5HT_{2C} receptor were assessed by incubation at 22° C. of a series of concentrations of test compound with intact CHO cells heterologously expressing the cloned human 5HT_{2C} receptor and measuring intracellular [Ca²⁺] by fluorimetry according to the method of Jerman et al., 2001, Eur. J. Pharmacol. 414:23-30. Antagonist effects were assessed by the ability of a series of concentrations of test compound to block the increase in intracellular [Ca²⁺] that occurred in the presence of 3.0 nM serotonin under the same conditions. EC₅₀ and IC₅₀ values were determined using standard methods.

Results. The results of the various binding and functional assays are summarized in Table 1, reproduced below.

TABLE 1
Affinity and Activity Data of (+/−), (+), and (−)-beloxepin for Various
Transporters and Receptors

| | NET | | 5HT_{2A} | | 5HT_{2B} | | 5HT_{2C} | |
|---|---|---|---|---|---|---|---|---|
| | Kᵢ, nM | IC₅₀, nM | Kᵢ, nM | IC₅₀, nM | Kᵢ, nM | IC₅₀, nM | Kᵢ, nM | IC₅₀, nM |
| (±) | 700 | 130 | 440 | 5200 | 1000 | >10,000 | 830 | >10,000 |
| | | | | antagonist | | antagonist | | antagonist |
| (−) | 390 | 120 | >10,000 | nd | >10,000 | nd | >10,000 | nd |
| (+) | 2920 | 1200 | 97 | 1600 | 170 | 690 | 84 | 7200 |
| | | | | antagonist | | antagonist | | antagonist |

nd = not determined

Racemic (±)-beloxepin is a weak inhibitor of NE reuptake (Kᵢ=700 nM) with marginal affinity at the 5HT and dopamine transporters (SERT: 27% inhibition at 10 μM; DAT: 16% inhibition at 10 μM). Racemic (±)-beloxepin was tested in binding assays with over 100 receptors, channels or transporters. From these experiments, it was determined that racemic (±)-beloxepin also binds with modest affinity to, and antagonizes, the 5HT_{2A}, 5HT_{2B} and 5HT_{2C} receptors. These data reveal that racemic (±)-beloxepin is a dual NRI/5HT_{2A,2B,2C} antagonist and that, quite surprisingly, the NRI activity is contributed virtually exclusively by the (−) enantiomer and the 5HT_{2A,2B,2C} antagonist activity virtually exclusively by the (+) enantiomer.

### Example 3

### (−)-Beloxepin is Effective in Treating Pain

Protocol. The antiallodynic activity of (−)-beloxepin was tested in vivo using the L5—Single Nerve Ligation model of non-nociceptive neuropathic pain as described in LaBuda & Little, 2005, J. Neurosci. Methods 144:175-181. The test animals were placed in a Plexiglas chamber (10 cm×20 cm×25 cm) and habituated for 15 minutes. The chamber was positioned on top of a mesh screen so that von Frey monofilaments could be presented to the plantar surface of both hindpaws. Measurement of tactile sensitivity for each hind paw were obtained using the up/down method (Dixon, 1980, Annu. Rev. Pharmacol. Toxicol. 20:441-462) with seven Frey monofilaments (0.4, 1, 2, 4, 6, 8 and 15 grams). Each trial started with a von Frey force of 2 grams delivered to the right hind paw for approximately 1-2 seconds and then the left hind paw. If there was no withdrawal response, the next higher force was delivered. If there was a response, the next lower force was delivered. This procedure was performed until no response was made at the highest force (15 grams) or until four stimuli were administered following the initial response. The 50% paw withdrawal threshold for each paw was calculated using the following formula: [Xth]log=[vFr]log+ky, where [vFr] is the force of the last von Frey used, k=0.2249 which is the average interval (in log units) between the von Frey monofilaments, and y is a value that depends upon the pattern of withdrawal responses (Dixon, 1980, supra). If an animal did not respond to the highest von Frey monofilament (15 grams), then the paw was assigned a value of 18.23 grams. Testing for tactile sensitivity was performed twice and the mean 50% withdrawal value assigned as the tactile sensitivity for the right and left paws for each animal.

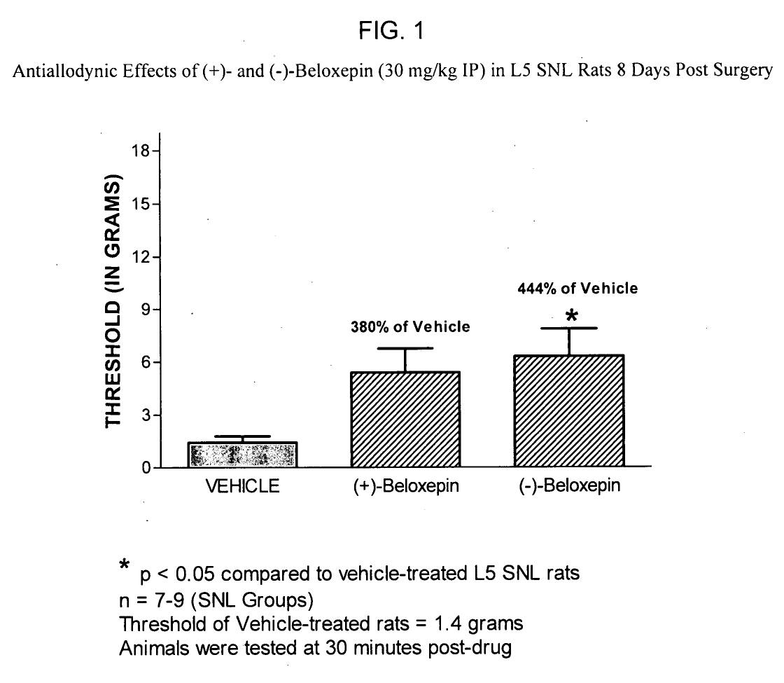

Results. The antiallodynic effects produced by (−)-beloxepin (30 mg/kg IP) and (+)-beloxepin (30 mg/kg IP) in L5 SNL rats 8 days post surgery are illustrated in FIG. 1. In this experiment, at 8 days post surgery, rats were treated with vehicle or beloxepin enantiomers (30 mg/kg IP) and tested for tactile allodynia at 30 min post treatment. As illustrated in FIG. 1, (−)-beloxepin produced significant antiallodynia (444% of the threshold of vehicle-treated L5 SNL rats). Although not statistically significant, (+)-beloxepin produced an antiallodynic effect that was comparable to that observed with (−)-beloxepin. No side effects were observed following treatment with either enantiomer.

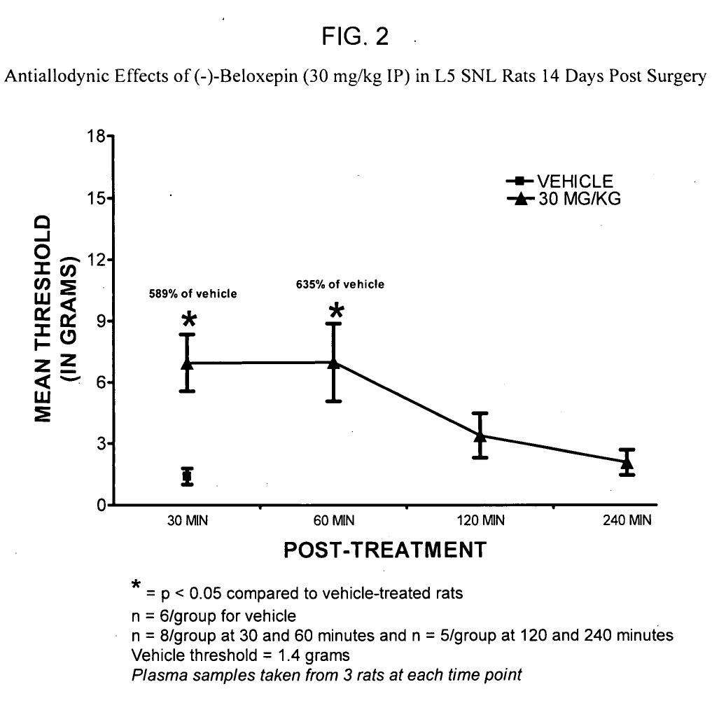

The antiallodynic effects produced by (−)-beloxepin (30 mg/kg IP) in L5 SNL rats 14 days post surgery are illustrated in FIG. 2. In this experiment, at 14 days post surgery, rats were treated with vehicle or (−)-beloxepin and tested for tactile allodynia at 30, 60, 120 and 240 min post treatment. Vehicle treated rats were tested at 30 min post treatment. As illustrated in FIG. 2, (−)-beloxepin produced significant antiallodynic effects at the 30 and 60 min time points, with a maximal efficacy corresponding to 635% of the threshold of vehicle-treated rats.

### Example 4

### (−)-Beloxepin Displayed Weak CYP2D6 Inhibitory Activity

Protocol. The inhibitory activities of (±), (−) and (+) beloxepin on cytochrome P450 function were tested using the methods of Chauret (Chauret et al., 2001, Drug Metabolism and Disposition, 29(9): 1196-2000), using 7-methoxy-4-(aminomethyl)-coumarin (MAMC) (Venhorst et al., 2000, European Journal of Pharmaceutical Sciences, 12(2):151-158) as substrate. The source of the enzyme was microsomes containing human recombinant CYP2D6 obtained from BD Bioscience. Conversion of MAMC to 7-hydroxy-4-(aminomethyl)coumarin was measured using a PerkinElmer Fusion with a 390 nm excitation filter and a 460 nm emission filter.

Results. The IC50s obtained for the compounds are provided in Table 3, below:

TABLE 3
CYP2D6 Isoenzyme IC₅₀s (nM)

| Compound | IC₅₀ (nM) |
|---|---|
| (±)-beloxepin | 536 |
| (−)-beloxepin | 4370 |
| (+)-beloxepin | 236 |

### Example 5

### (−)-Beloxepin is Effective in an Animal Model of Neuropathic Pain (Rat L5 SNL Model)

Protocol. A time course experiment was performed with (−)-beloxepin (60 mg/kg PO) in L5 SNL rats at 7 days post-surgery. Rats were tested at 30, 60, 120, and 240 minutes post-drug.

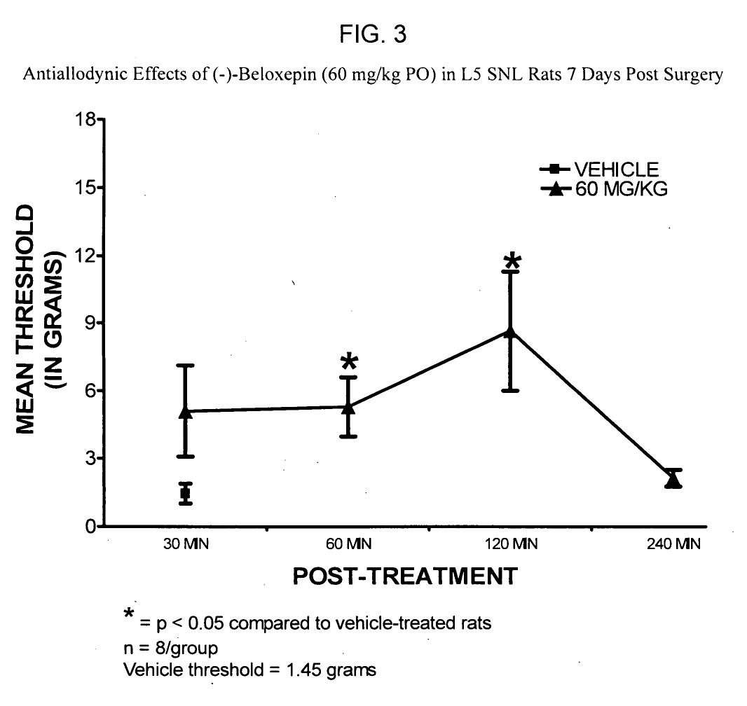

Results. The (−)-beloxepin enantiomer produced significant antiallodynic effects at the 60 and 120 minute time points, as illustrated in FIG. 3.

Protocol. A time course experiment was also performed with (+)-beloxepin (60 mg/kg PO) in L5 SNL rats at 14 days post-surgery. Rats were tested at 30, 60, 120, and 240 minutes post-drug.

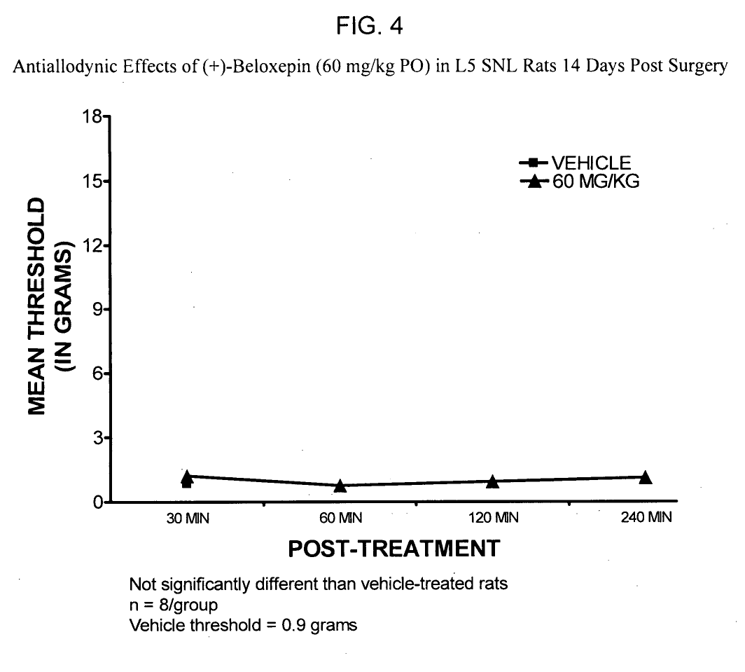

Results. The (+)-beloxepin enantiomer did not produce significant antiallodynic effects at any time point (FIG. 4).

### Example 6

### (−)-Beloxepin is Effective in an Animal Model of Post-Operative Pain (Rat Hindpaw Incisional Pain Model)

Protocol. A time course experiment was also performed with (−)-beloxepin in the hindpaw incision model. At 24 hours post-surgery, rats received vehicle or (−)-beloxepin (30 mg/kg IP). Rats were tested for tactile allodynia at 30, 60, 120 and 240 minutes after administration of (−)-beloxepin.

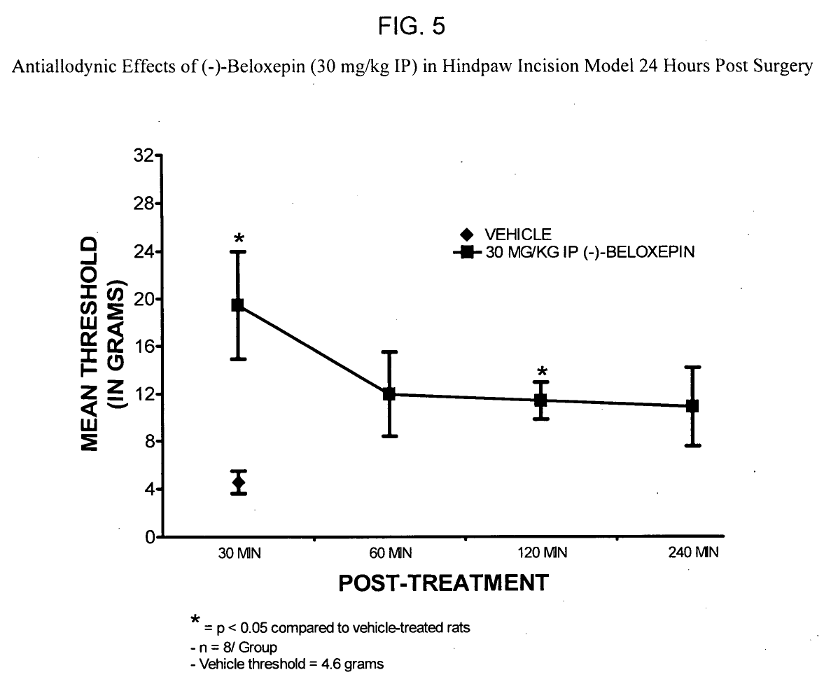

Results. As illustrated in FIG. 5, (−)-beloxepin produced a significant antiallodynic effect at the 30 and 120 minute time point (maximum hindpaw withdrawal threshold ˜19 grams or 426% of the threshold value for vehicle treated rats at the 30 minute time point). The antiallodynic effect produced by (−)-beloxepin at the 30 minute, but not 120 minute, time point is considered robust.

Protocol. Another time course experiment was performed with (+)-beloxepin in the hindpaw incision model. At 24 hours post-surgery, rats received vehicle or (+)-beloxepin (30 mg/kg IP). Rats were tested for tactile allodynia at 30, 60, 120 and 240 minutes after administration of (+)-beloxepin.

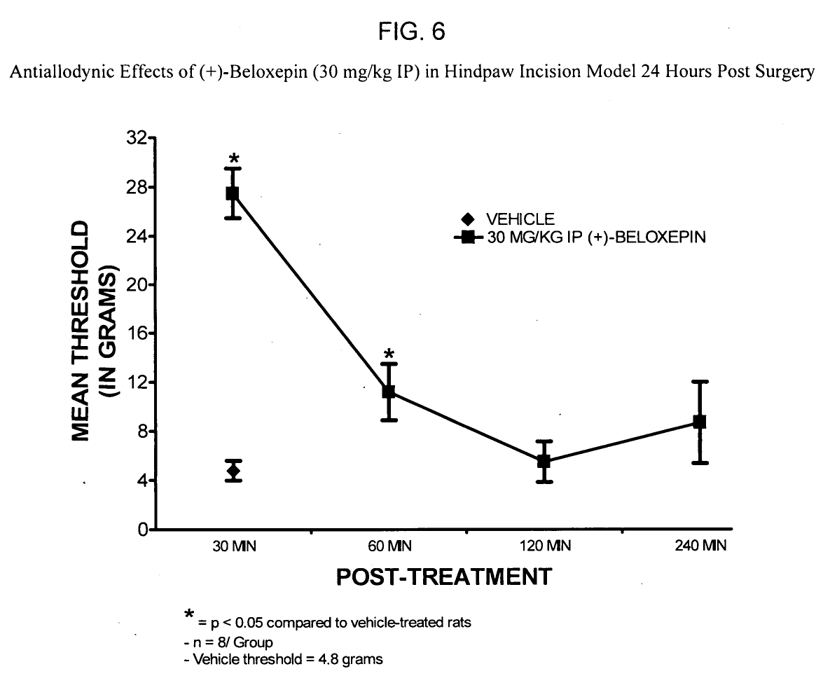

Results. As illustrated in FIG. 6, (+)-beloxepin produced a significant antiallodynic effect at the 30 and 60 minute time points (maximum hindpaw withdrawal threshold ˜28 grams at the 30 minute time point). The antiallodynic effect produced by (+)-beloxepin in this assay is considered very robust and comparable to the effect observed with racemic beloxepin at the 30 minute time point.

### Example 7

### (−)-Beloxepin is Effective in an Animal Model of Nociceptive Pain (Rat L5 SNL Model)

Protocol. Rats were treated with vehicle, 30 mg/kg IP of (−)-beloxepin, or 30 mg/kg IP of (+)-beloxepin and then were placed on a hot plate (50° C.) 30, 60, or 120 minutes after administration of the vehicle or beloxepin enantiomer. Rats treated with 3 mg/kg SC of morphine and tested 30 min post-treatment were used as a positive control in these experiments.

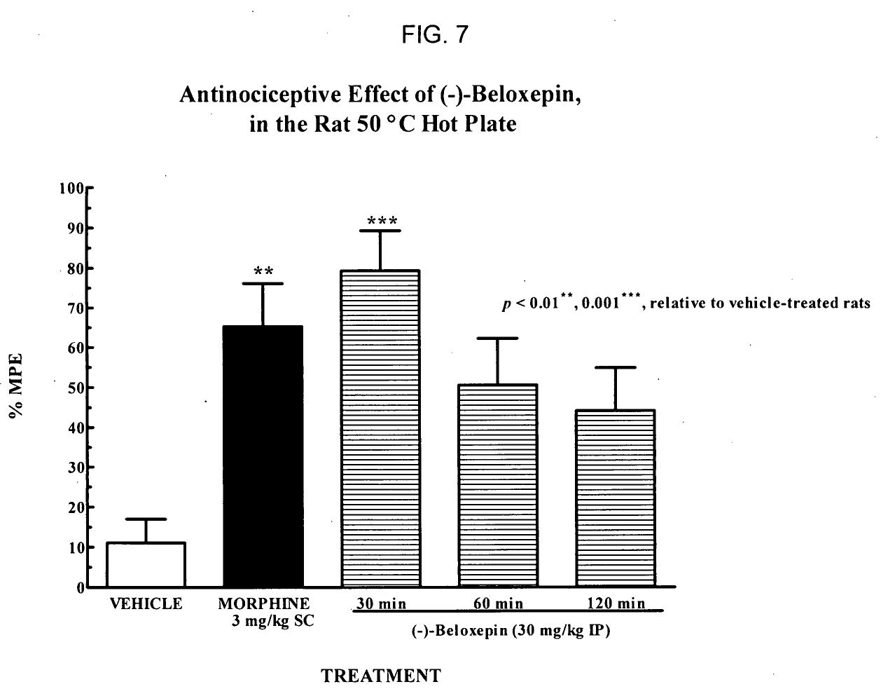

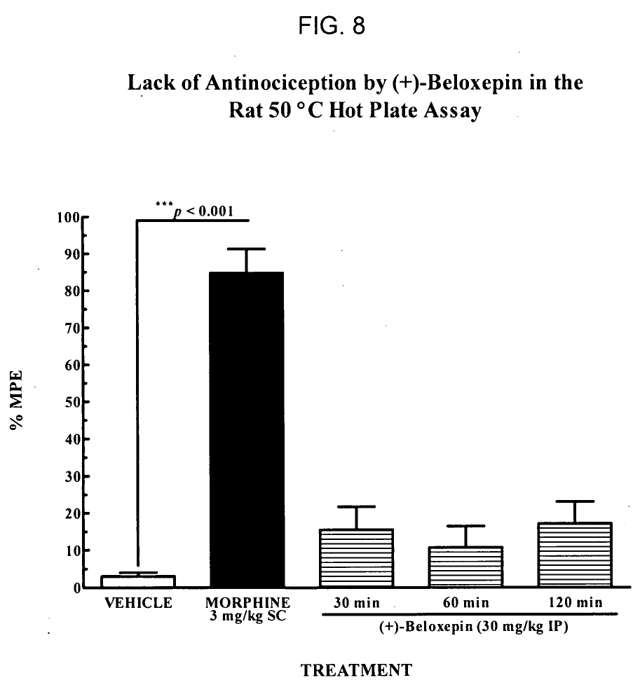

Results. Treatment with morphine (3 mg/kg SC) resulted in a level of antinociception of 61±7% MPE in these experiments. Testing of the (−)- and (+)-enantiomers of beloxepin in the rat 50° C. hot plate assay demonstrated enantioselective effects, as illustrated in FIG. 7 ((−)-beloxepin) and FIG. 8 ((+)-beloxepin). (−)-Beloxepin displayed robust antinociceptive activity at 30, 60, and 120 minutes after treatment, with peak antinociception of 79±10% MPE at 30 min post-treatment (FIG. 7). In this experiment, morphine (3 mg/kg SC) treatment produced 65±11% MPE. In contrast, no antinociception was observed in rats treated with (+)-beloxepin (FIG. 8), with % MPE that were not significantly different from vehicle-treated rats with % MPE values ranging 10-17%. In morphine-treated rats the level of antinociception was 85±7% MPE.

While various specific embodiments have been illustrated and described, it will be appreciated that various changes can be made without departing from the spirit and scope of the invention(s).

All publications, patents, patent applications and other documents cited in this application are hereby incorporated by reference in their entireties for all purposes to the same extent as if each individual publication, patent, patent application or other document were individually indicated to be incorporated by reference for all purposes.

What is claimed is:

1. Beloxepin enriched in the (−) enantiomer.

2. Substantially enantiomerically pure (−)-beloxepin.

3. Enantiomerically pure (−)-beloxepin.

4. A composition comprising beloxepin and an excipient, carrier and/or diluent, wherein the beloxepin is enriched in the (−) enantiomer.

5. The composition of claim 4 in which the beloxepin is substantially enantiomerically pure (−)-beloxepin.

6. The composition of claim 4 in which the beloxepin is enantiomerically pure (−)-beloxepin.

7. The composition of any one of claims 4-6 which is formulated for pharmaceutical use.

8. The composition of claim 7 which is formulated for oral administration to humans.

9. The composition of claim 7 which is formulated for parenteral administration to humans.

10. A method of treating pain in a mammal, comprising administering to the mammal an amount of beloxepin which is enriched in the (−) enantiomer effective to treat the pain.

11. A method of treating pain in a mammal, comprising administering to the mammal an amount of substantially enantiomerically pure (−)-beloxepin effective to treat the pain.

12. A method of treating pain in a mammal comprising administering to the mammal an amount of enantiomerically pure (−)-beloxepin effective to treat the pain.

13. A method of treating pain in a mammal, comprising administering to the mammal an amount of a composition comprising beloxepin effective to treat the pain, wherein the beloxepin is enriched in the (−) enantiomer.

14. The method of claim 13 in which the beloxepin is substantially enantiomerically pure (−)-beloxepin.

15. The method of claim 13 in which the beloxepin is enantiomerically pure (−)-beloxepin.

16. The method of claim 13 in which the composition is formulated for oral administration to humans.

17. The method of any one of claims 9-16 in which the pain is selected from nociceptive pain, non-nociceptive pain, acute pain, chronic pain, inflammatory pain, pain associated with irritable bowel syndrome, pain associated with rheumatoid arthritis, pain associated with cancer, pain associated with osteoarthritis, neuropathic pain, post-herpetic neuralgia (PHN), trigeminal neuralgia, focal peripheral nerve injury, anesthesia clolorosa, central pain, post-stroke pain, pain due to spinal cord injury, pain associated with multiple sclerosis, peripheral neuropathy, diabetic neuropathy, inherited neuropathy and acquired neuropathy.

18. The method of claim 17 in which the mammal is a human.

19. The method of claim 18 in which the pain is neuropathic pain.

20. A method of inhibiting NE reuptake, comprising contacting a NE transporter with an amount of beloxepin effective to inhibit reuptake of NE, wherein the beloxepin is enriched in the (−) enantiomer.

21. The method of claim 20 in which the beloxepin is substantially enantiomerically pure (−)-beloxepin.

22. The method of claim 20 in which the beloxepin is enantiomerically pure (−)-beloxepin.

23. The method of any one of claims 20-22 which is practiced in vitro.

24. The method of any one of claims 20-22 which is practiced in vivo.

25. A method of inhibiting reuptake of NE in a human, comprising administering to a human an amount of a composition comprising beloxepin effective to inhibit NE reuptake, wherein the beloxepin is enriched in the (−) enantiomer.

26. The method of claim 25 in which the beloxepin is substantially enantiomerically pure (−)-beloxepin.

27. The method of claim 25 in which the beloxepin is enantiomerically pure (−)-beloxepin.

28. The method of any one of claims 25-27 in which the composition is administered orally.

29. The method of any one of claims 25-27 in which the composition is administered parenterally.

30. A method of treating a disorder in a patient that is responsive to treatment with an NRI compound, comprising administering to the patient an amount of a composition comprising beloxepin effective to treat the disease or disorder, wherein the beloxepin is enriched in the (−) enantiomer.

31. The method of claim 30 in which the beloxepin is substantially enantiomerically pure (−)-beloxepin.

32. The method of claim 30 in which the beloxepin is enantiomerically pure (−)-beloxepin.

33. The method of any one of claims 30-32 in which the disorder responsive to treatment with an NRI compound is selected from mood disorders, cognitive disorders, psychotic disorders, anxiety disorders, personality disorders, eating disorders, impulse disorders, tic disorders, pre-menstrual syndrome or dysphoria and fibromyalgia.

34. The method of claim 33 in which the disorder is selected from the group consisting of depression, obsessive-compulsive disorder, anorexia nervosa, bulimia nervosa, trichotillomania, insomnia related to opioid withdrawal, and attention deficit hyperactivity disorder.
